(19) United States
(12) Patent Application Publication
(10) Pub. No.: US 2005/0244335 A1
(43) Pub. Date: Nov. 3, 2005

(54) Methods and compositions related to increasing antitumor activity of chemotherapeutic agents

(76) Inventors: John J. Rinehart, Lexington, KY (US); Hui Wang, Birmingham, AL (US); Ruiwen Zhang, Marietta, GA (US)

Correspondence Address: NEEDLE & ROSENBERG, P.C., SUITE 1000, 999 PEACHTREE STREET, ATLANTA, GA, 30309-3915, US

(21) Appl. No.: 11/120,644

(22) Filed: May 3, 2005

Related U.S. Application Data
(60) Provisional application No. 60/567,766, filed on May 3, 2004.

(51) Int. Cl. A61K 49/00

(52) U.S. Cl. 424/9.2

(57) ABSTRACT

Disclosed are methods and compositions related to increasing antitumor activity of chemotherapeutic agents.

This application claims priority to U.S. provisional application No. 60/567,766, filed on May 3, 2005. The aforementioned application is herein incorporated by this reference in their entirety.

### BACKGROUND OF THE INVENTION

DNA and RNA interactive chemotherapeutic agents remain the most effective and widely used approach in medical cancer therapy. Two major problems remain to be overcome in order to improve the therapeutic effectiveness and safety profiles of cancer chemotherapy. First, most cancer chemotherapeutic agents have severe side effects (Demetri, 1995), including bone marrow suppression, the major dose-limiting toxicity of many chemotherapeutic agents (Mackal, 2000). To reverse chemotherapy-induced hematotoxicity, post-chemotherapy administration of hematopoietic growth factors such as granulocyte colony stimulating factor (G-CSF) and granulocyte-macrophage colony stimulating factor (GM-CSF) is frequently employed (Griffin, 1997). However, this approach has several important limitations: relative ineffectiveness, expense, and failure to prevent genomic alterations and hematopoietic progenitor depletion. In contrast, pretreatment of hematopoietic growth factors may offer preventive benefits. For instance, pretreatment strategies to protect hematopoietic progenitors from chemotherapeutic agent-induced toxicity have been tested in animal models, including the use of corticosteroids (Kriegler, 1994; Joyce, 1977; Rinehart, 1994, 1995, 1997), and cytokines (Aman, 1994; Chudgar, 1995; Cashman, 1990; Futami, 1990; Dunlop, 1992; Grzegorzewski, 1994). GM-CSF (Vadhan, 1992; Broxmeyer, 1994; Janick, 1993; Aglietta, 1993) and amifostine (Betticher, 1993) have been demonstrated to have hematoprotective effects in cancer patients receiving chemotherapy. However, there are concerns that pretreatment with corticosteroids may compromise therapeutic efficiency of cancer chemotherapeutic agents (Gorman, 2000).

The second major problem associated with cancer chemotherapy is drug resistance. The investigations of tumor resistance to these agents have nearly always focused on cellular and molecular mechanisms. However, some evidence has suggested that physiological mechanisms may also play an important role in tumor resistance to chemotherapeutic agents. For example, Teicher et al demonstrated that resistance of murine tumors to chemotherapeutic agents, which was developed in vivo, was not associated with in vitro resistance but with a decreased accumulation of drug in tumor in vivo (Teicher, 1990).

What is needed in the art is an effective method of increasing antitumor activity of cytotoxic agents, and decreasing host hematotoxicity of cytotoxic agents.

### SUMMARY

Disclosed herein are methods of screening for an agent that increases the efficacy of an antineoplastic chemotherapeutic agent and reduces the hematotoxicity of the chemotherapeutic agent.

Also disclosed are methods of treating a subject with cancer comprising administering to the subject the compositions disclosed herein.

### BRIEF DESCRIPTION OF THE DRAWINGS

The accompanying drawings, which are incorporated in and constitute a part of this specification, illustrate embodiments of the invention and together with the description, serve to explain the principles of the invention.

FIG. 1 shows dexamethasone (DEX) decreases host toxicity of carboplatin chemotherapy in CD-1 mice. Animals were randomly divided into multiple treatment and control groups (10 mice/group). DEX (s.c., 0.1 mg/mouse/day for 5 days) or saline (in controls) was given prior to or after single i.p. injection of carboplatin (600 mg/m² or 200 mg/kg).

FIG. 2 shows dexamethasone prevents hemotopoietic toxicity of carboplatin chemotherapy in CD-1 mice. Animals were randomly divided into multiple treatment and control groups (10 mice/group). Various doses of DEX (s.c., 0.05, 0.1, 0.3 mg/mouse/day for 5 days) were given prior to single i.p. dose of carboplatin (360 mg/m² or 120 mg/kg) or post-chemotherapy. The data presented are nadirs of granulocyte counts, expressed as percentage of untreated control, following carboplatin treatment (single dose, 360 mg/m²) on day 14.

FIG. 3 shows the pharmacokinetics of carboplatin in CD-1 mice. Animals were pre-treated with DEX (s.c., 0.1 mg/day/mouse for 5 days) or saline (as controls) and given carboplatin at a single IV dose of 60 mg/kg (180 mg/m²). Plasma and tissue samples were taken at various times up to 24 hr. Carboplatin was analyzed by HPLC. Panel A: Plasma; Panel B: Liver; Panel C: Spleen; Panel D: Bone Marrow. Panel E: Comparison of AUC of carboplatin in plasma and various tissues. AUC: Area under the time-concentration curve; B.M.: Bone marrow.

FIG. 4 shows the pharmacokinetics of gemcitabine in CD-1 mice. Animals were pre-treated with DEX (0.1 mg/day/mouse for 5 days) or saline (as controls) and given [³H]-gemcitabine at a single iv dose of 160 mg/kg. Panel A: Plasma; Panel B: Liver; Panel C: Spleen; Panel D: Bone Marrow. Panel E: Comparison of AUC of gemcitabine in plasma and various tissues. AUC: Area under the time concentration curve; B.M.: Bone marrow.

FIG. 5 shows the effects of dexamethsone (DEX) on antitumor activity of carboplatin (A) and gemcitabine (B) chemotherapy in nude mice bearing human colon cancer LS174T xenografts. Animals, randomly divided into various treatment and control groups (5 mice/group), were pre-treated with DEX (s.c., 0.1 mg/day for 5 days, days −4 to 0) or saline (as control). Carboplatin was administered intraperitoneally (i.p.) at a single dose of 120 mg/kg (360 mg/m²) on day 0. Gemcitabine was given twice at an i.p. dose of 160 mg/kg (480 mg/m²) on days 0 and 4. Panel C: Photographs of tumors removed on day 12. Random tumors (3) from each group are shown.

FIG. 6 shows the effects of dexamethsone (DEX) on antitumor activity of carboplatin and gemcitabine chemotherapy in nude mice bearing human lung cancer A549 or H1299 xenografts. Animals, randomly divided into various treatment and control groups (5 mice/group) were treated using the same protocol as above (FIG. 1). Panels A and B: A549 model treated with carboplatin (A) or gemcitabine (B); Panel C: H1299 model treated with carboplatin.

FIG. 7 shows the effects of dexamethsone (DEX) on antitumor activity of carboplatin and gemcitabine chemotherapy in nude mice bearing human breast cancer MCF-7 or MDA-MB-468 xenografts. Animals, randomly divided into various treatment and control groups (5 mice/group) were treated using the same protocol as above (FIG. 1). Panels A and B: MCF-7 model treated with carboplatin (A) or gemcitabine (B); Panel C: MDA-MB-468 model treated with carboplatin.

FIG. 8 shows the effects of dexamethasone (DEX) on antitumor activity of carboplatin (A) or gemcitabine (B) chemotherapy and combination of carboplatin and gemcitabine (C) chemotherapy in nude mouse xenograft model of human glioma U87-MG. Animals, randomly divided into various treatment and control groups (5 mice/group), were pre-treated with DEX (s.c., 0.1 mg/day for 5 days) or saline (as controls). Carboplatin was administered intraperitoneally (i.p.) at a dose of 120 mg/kg or 360 mg/m² on day 0. Gemcitabine was given at a single i.p. dose of 160 mg/kg (480 mg/m²) on day 0.

FIG. 9 shows immunohistochemistry with CD45 staining of A549 Tumor Xenografts. Mice were sacrificed on day 24 of study (see FIG. 2). The CD45 antibody was a purified rat anti-mouse CD45 (leukocyte common antigen, Ly-5) monoclonal antibody from BD Biosciences. A: Positive control (mouse spleen, 100×); B: Positive control (mouse spleen, 1000×); C: Saline control; D: Dexamethasone control; E: Carboplatin alone; F: Dexamethasone plus Carboplatin. Scant numbers of CD45 cells were seen and there was no difference between tumors from the four groups.

FIG. 10 shows pharmacokinetics of carboplatin in nude mice bearing human colon cancer LS174T xenografts. Animals were pre-treated with Dexamethasone (s.c., 0.1 mg/day/mouse for 5 days) or saline (as controls) and given carboplatin at a single IV dose of 60 mg/kg (180 mg/m²). Plasma and tissue samples were taken at various times up to 24 hr. Carboplatin was analyzed by HPLC. Panel A: Plasma; Panel B: Tumor; Panel C: Spleen; Panel D: Bone Marrow. Panel E: Comparison of AUC of carboplatin in plasma and various tissues. AUC: Area under the time-concentration curve; B.M.: Bone marrow.

FIG. 11 shows pharmacokinetics of carboplatin in nude mice bearing human lung cancer A549 xenografts. Animals were pre-treated with DEX (0.1 mg/day/mouse for 5 days) or saline (as controls) and given carboplatin at a single iv dose of 60 mg/kg. Carboplatin was analyzed by HPLC. Panel A: Plasma; Panel B: Tumor; Panel C: Spleen; Panel D: Bone Marrow. Panel E: Comparison of AUC of carboplatin in plasma and various tissues. AUC: Area under the time-concentration curve; B.M.: Bone marrow.

FIG. 12 shows time-concentration profiles of gemcitabine in nude mice bearing human cancer H1299 xenografts. Animals were pre-treated with DEX (0.1 mg/day/mouse for 5 days) or saline (as controls) and given [³H]-gemcitabine at a single iv dose of 160 mg/kg. Panel A: Plasma; Panel B: Tumor; Panel C: Spleen; Panel D: Bone Marrow. Panel E: Comparison of AUC of gemcitabine in plasma and various tissues. AUC: Area under the time concentration curve; B.M.: Bone marrow.

FIG. 13A shows a mouse prostate cancer TRAMP model. The treatments started on mean tumor size reached 197 mg. Oligo #2055, #2092 (1 mg/kg): s.c., 3 doses/week for 4 weeks. Doxil (10 mg/kg): iv, on day 4 for CpG study and on day 0 for DEX study. FIGS. 13B and 13C show the effects of dexamethasone (DEX) on antitumor activity (B) and body weight (C) of ADR chemotherapy in BALB/C mice bearing mouse breast cancer 4T1. Animals randomly divided into various treatment and control groups (5 mice/group) were pretreated with DEX (s.c., 0.1 mg/day for 5 days, days −4 to 0) or saline (as control). ADR was administered i.v. at a single dose of 10 mg/kg (30 mg/m2) on day 0. Tumor mass was expressed as mean ±SD.

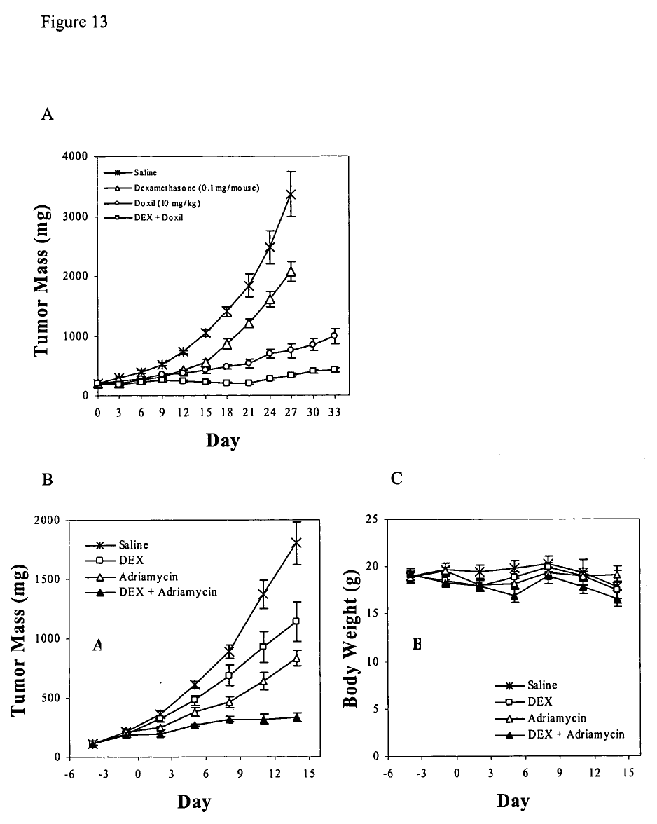

### DETAILED DESCRIPTION OF THE INVENTION

The present invention may be understood more readily by reference to the following detailed description of preferred embodiments of the invention and the Examples included therein and to the Figures and their previous and following description.

As used in the specification and the appended claims, the singular forms “a,” “an” and “the” include plural referents unless the context clearly dictates otherwise. Thus, for example, reference to “a small molecule” includes mixtures of one or more small molecules, and the like.

Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

The terms “higher,” “increases,” “elevates,” or “elevation” refer to increases above control levels. The terms “low,” “lower,” “reduces,” or “reduction” refer to decreases below basal levels, e.g., as compared to a control. For example, basal levels are normal in vivo levels prior to, or in the absence of, addition of an agent such as dexamethasone.

There are considerable interests in the development of clinical strategies to prevent or reverse the side effects of cancer chemotherapy (Kaufman 1996; Demetri, 1995). Most clinically used cancer chemotherapeutics are DNA interactive agents with varying mechanisms of action. Amongst many side effects of these chemotherapeutic agents is bone marrow suppression. These agents cause DNA damage to the lymphohematopoietic precursor, decreasing blood cellular elements (Demetri, 1995), which is seen as blood cytopenias (decrease in circulating platelets, white and red blood cells) in the clinic (Demetri, 1995; Mackal, 2000). In clinical practice, hematopoietic growth factors such as granulocyte colony stimulating factor (G-CSF) and granulocyte-macrophage colony stimulating factor (GM-CSF) are frequently used after chemotherapy with the hope to reduce hematotoxicity (Ganser, 1996; Griffin, 1997). These cytokines have been used in the treatment of primary bone marrow failure states and after myelosuppressive chemotherapy or radiotherapy. Most studies with G-CSF and GM-CSF showed acceleration of granulocyte recovery after chemotherapy and radiotherapy, resulting in a reduction of infectious risks, a shortening of drug- and radiation-induced myelosuppression, and a higher chemotherapy dose intensity (Ganser, 1996; Griffin, 1997). In addition, post-therapy administration of hematopoietic growth factors is expensive and fails to prevent genomic damage and hematopoietic progenitor depletion.

Alternatively, administration of hematopoietic growth factors prior to chemotherapy or radiotherapy can offer preventive benefits to cancer patients. Several approaches have been tested in animal models, including the use of corticosteroids (Joyce, 1997; Kriegler, 1994; Rinehart; 1994; Rinehart, 1995; Rinehart, 1997), cytokines (Aman, 1994; Chudgar, 1995; Cashman, 1990; Futami, 1990; Dunlop, 1992, Grzegorzewski, 1994), and scavengers of chemotherapeutic agents or their metabolites (Peters, 1995), and vectors to introduce chemotherapy resistance into stem cells (Podda, 1992). It is believed that corticosteroids have the ability to suppress the production of growth factors and cytokines and are thus implicated in the negative regulation of hematopoiesis. In a study with mouse models, Kriegler et al (Kriegler, 1994) demonstrated that the corticosteroids prednisolone and dexamethasone (DEX), effectively protected progenitor cells against several chemotherapeutic agent 5-fluorouracil, with better protective effects being observed with DEX than prednisolone. In murine models, administration of corticosteroids prior to chemotherapy reduced carboplatin-induced hematotoxicity (Rinehart, 1994; Rinehart, 1995; Rinehart; 1997). These findings indicate that the currently clinical used treatment schedules of corticosteroids during cancer therapy need to be reexamined to obtain the maximum benefits for cancer patients with chemotherapy or radiotherapy. Thus far, DEX, GM-CSF (Vadhan-Raj, 1992; Broxmeyer, 1994; Janik, 1993; Aglietta, 1993), and amifostine (Betticher, 1995), have been shown to have hematoprotective effects in cancer patients receiving chemotherapy.

Carboplatin represents a new generation of platinum anticancer compounds, shares some of the therapeutic advantages of cisplatin, but without a significant incidence of the dose-limiting neurotoxicity and nephrotoxicity which is experienced with cisplatin (Duffull, 1997). However, its use is associated with dose-limiting bone marrow suppression. In preclinical models, pretreatment with corticosteroids remarkably reduced carboplatin-induced hematotoxicity (Rinehart, 1994; Rinehart, 1995; Rinehart; 1997). In a Phase I clinical trial, the hematoprotective effect of dexamethasone (DEX) was demonstrated in patients with metastatic cancer who were treated with carboplatin and ifosfamide. Gemcitabine has been established as a new standard for the treatment of pancreatic cancer (Heineman, 2002). It has been shown to improve clinical benefits including response, time to progression, and survival, compared with other chemotherapeutic agents such as 5-fluorouracil. In clinical trial, the combination of cisplatin and gemcitabine significantly improved tumor response and time to progression as compared with gemcitabine alone (Heineman, 2002). The effects of dexamethasone on the plasma and tissue pharmacokinetics of carboplatin in experimental animals were therefore examined (Example 1).

The rationale to develop chemoprotective approaches was to alter the microenvironment of critical tissues/organs that are susceptible to unwanted toxicity from chemotherapeutic agents. Murine models were employed to determine the biological effects on carboplatin/gemcitabine and pharmacokinetic mechanisms of pretreatment with dexamethasone. The results from the present study demonstrated at least four points. First, in a dose-dependent manner, pretreatment with dexamethasone significantly reduced the mortality of carboplatin therapy in CD-1 mice. Second, in a dose-dependent manner, DEX pretreatment significantly reversed carboplatin-induced hematotoxicity in CD-1 mice, which can be responsible for the reduction of carboplatin-associated mortality. Third, pretreatment with dexamethasone significantly decreased the carboplatin concentrations in spleen and bone marrow. Fourth, pretreatment with dexamethasone also significantly decreased the gemcitabine concentrations in spleen and bone marrow as seen with carboplatin, although the two compounds have relatively different patterns of tissue distribution.

To illustrate the protective effects of pretreatment with dexamethasone on chemotherapy induced toxicity, CD-1 mice were used. The results demonstrated that pre-treatment with dexamethasone significantly reduced the mortality of carboplatin administered alone or in combination with gemcitabine. In Example 1, it was demonstrated that pretreatment with dexamethasone had a hematopoietic protection effect on carboplatin-based chemotherapy in mice; this effect was dose- and schedule-dependent. It has been shown that pretreatment with corticosteroid protected experimental animals from chemotherapy-induced hematopoietic toxicity (Joyce, 1997; Kriegler, 1994; Rinehart; 1994; Rinehart, 1995; Rinehart, 1997). For example, Joyce and Chervenick (Joyce, 1997) demonstrated that pre-treatment of mice with a single dose of corticosteroid reduced bone marrow depletion of granulocyte-macrophage colony forming units (CFU-GM) and protected mice from intravenous bacterial challenge following a sub-lethal dose of chemotherapy with cyclophosphamide. They also examined post-chemotherapy bone marrow CFU-GM for sensitivity to high-specific activity ³H-thymidine and concluded that a lower fraction of residual CFU-GM was in S-phase after treatment with corticosteroids and cyclophosphamide, compared to treatment with cyclophosphamide alone (Joyce, 1997).

Induction of stem cell resistance to DNA interactive agents occurs at the cellular level. Pharmacokinetic factors are involved in the process. The decreases in spleen and bone marrow uptake of carboplatin and gemcitabine in mice pretreated with dexamethasone can explain the mechanisms responsible for reduced hematotoxicity of carboplatin/gemcitabine. The reasons for decreased tissue uptake can be associated with drug redistribution and decrease in spleen tissue mass and cell density following dexamethasone treatment. Matsukado and coworkers demonstrated that dexamethasone decreased brain uptake of carboplatin (Matsukado, 1997).

Since there is no significant overlap of side effects between carboplatin and gemcitabine, the combination of the both can offer clinical benefits to cancer patients. Pretreatment with dexamethasone can be a powerful approach to prevent carboplatin-associated bone marrow toxicity and reduce tissue levels of both drugs in spleen and bone marrow.

Dexamethasone can also be used to enhance in vivo antitumor activities of carboplatin, gemcitabine or the combination of both. Six nude mouse models of human cancers showed significant antitumor activity (Example 2). There are reports demonstrating in vivo antitumor activity of DEX in epithelial cell cancers (Braunschweiger, 1983, 1984). DEX can decrease tumor secretion or tumor associated-cell secretion of tumor growth factors. For example, Nishimura et al demonstrated that DEX inhibited the growth of human prostate cancer DU145 xenografts in nude and SCID mice, possibly through the disruption of the NF-kappaB-IL-6 pathway (Nishimura, 2001). In addition, DEX can enhance tumor apoptosis by inhibiting NF-kB activity by at least two mechanisms: DEX induces translation of I-kB and CILZ which inhibit NF-kB translation to the nucleus (Auphan, 1995) and interaction with transcription sites respectively (Berrebi, 2003). NF-kB induces transcription of multiple anti-apoptotic proteins in stressed cell (Wang, 1998).

Dexamethasone enhancement of anti-tumor effects of carboplatin and gemcitabine can be explained in several ways. Tumors exhibit multiple physiological abnormalities including markedly abnormal tortuous vasculature characterized by decreased blood flow, decreased lymphatic and promiscuous movement of large molecular weight solutes through abnormal inter-endothelial pores (Carmeliet, 2000; Braunschweiger, 1986; Jain, 1987; Hashizume, 2000). These abnormalities result in elevated tumor interstitial fluid pressure (TIFP) (Rofstad, 2002) and volume (Kerbel, 2002), which paradoxically in turn, reduces the movement of agents that are transiently present in the plasma into the tumor interstitial fluid space as predicted by Darcy's Law (Jain, 2001). The mechanisms of induction of “leaky” inter-endothelial pores are probably multifactorial. Tumor endothelial cells are structurally abnormal (Braunschweiger, 1986). Moreover, tumor endothelial cells are exposed to several cytokines such as VEGF, IL-1, IL-8, and TGF-β, which alter both normal and tumor vasculature to allow movement of solutes and fluid into the interstitial space (Ono, 1999; Coussens, 2002). The origin of those cytokines is clearly multiple and may include tumor cells and tumor infiltrating cells such as macrophages (Ono, 1999; Coussens, 2002; Torisu, 2000; Barbera-Guillem, 2002; Underwood, 1999). Several studies have shown that glucocorticosteroids reduce inter-endothelial pore size in pure, normal endothelial cell cultures in vitro (Underwood, 1999) and inhibit secretion of inflammatory cytokines from many cells, including macrophages (Auphan, 1995; Yamamoto, 2001). Braunschweiger and Schiffer demonstrated that treatment with dexamethasone (DEX) of mice bearing autochronous tumors decreased the movement of large molecular weight molecules into the tumors and the tumor interstitial volume (Braunschwieger, 1986). As predicted by those studies, treatment of murine-human colon cancer LS174T xenografts with dexamethasone reduced elevated TIFP (Kristjansen, 1993). Using the same model (LS174T) it has been demonstrated that dexamethasone increases drug uptake and improves therapeutic effectiveness of carboplatin and gemcitabine. Therefore, dexamethasone can enhance antitumor effects of carboplatin and gemcitabine by decreasing in tumors the promiscuous inter-endothelial loss of solutes and water into the interstitial space, thus decreasing elevated interstitial volume and pressure in the interstitial space. Decreased tumor interstitial fluid pressure, in turn, allows for increased drug movement into tumors and improved antitumor effects.

Furthermore, pretreatment with dexamethasone decreases movement of carboplatin and gemcitabine into normal tissue and increase movement of these drugs into tumors, as discussed above. Since in normal tissue IFP is already low (−5 to 0 mmHg vs 5-100 mmHg in tumors) the dominant effect in normal tissue is retardation of solute movement (i.e., drug) into bone marrow and spleen. Abnormal tumor vasculature allows promiscuous movement of solutes and water into the interstitial space, however, movement of carboplatin or gemcitabine into tumor decreased. Solutes (and water) which are continuously in plasma eventually equilibrate with the interstitial space and increase IFP. Solutes transiently in plasma (e.g., drugs) cannot move against the pressure gradient to achieve equilibrium levels during the relatively short time in the plasma. These studies show that pretreatment of patients with dexamethasone can decrease hematotoxicity and increase antitumor effects of carboplatin or gemcitabine. Dexamethasone decreased carboplatin induced hematotoxicity. In patients receiving dexamethasone the incidence of partial or complete responses was higher than in patients not receiving dexamethasone.

Disclosed are methods of screening for an agent that increases the efficacy of an antineoplastic chemotherapeutic agent and reduces the hematotoxicity of the chemotherapeutic agent. These methods can include the steps of: contacting a test tumor cell with the chemotherapeutic agent and the agent to be screened and detecting a reduction in cell division or an increase in cell death as compared to a control tumor cell contacted with the chemotherapeutic agent in the absence of the agent to be screened, a reduction in cell division or an increase in cell death indicating an agent that increases in the efficacy of the chemotherapeutic agent; and contacting a test hematopoietic cell with the chemotherapeutic agent and the agent to be screened and detecting an increase in cell division or a reduction in cell death as compared to a control hematopoietic cell contacted with the chemotherapeutic agent in the absence of the agent to be screened, an increase in cell division or a decrease in cell death indicating an agent that reduces the hematotoxicity of the chemotherapeutic agent. These methods can occur in vivo or in vitro or ex vivo, for example.

The increase in efficacy of the chemotherapeutic agent can comprise an increase in uptake of the chemotherapeutic agent by the test tumor cell as compared to the control tumor cells. Furthermore, the reduction in hematotoxicity can comprise a decrease in uptake of the chemotherapeutic agent by the test hematopoietic cell as compared to the control hematopoietic cell.

The tumor cell can be a solid tumor cell, can be a cell from a tumor cell line, or can be a hematopoietic cell such as a splenic cell or a bone marrow cell, for example.

The test tumor cell or the test hematopoietic cell can be contacted with the agent to be screened prior to contact with the chemotherapeutic agent. In one embodiment, the test tumor cell or the test hematopoietic cell can be contacted with the agent to be screened daily for one hour, two hours, three hours, four hours, six hours 12 hours, or one, two, three, four, five, six, or seven days prior to contact with the chemotherapeutic agent. The test tumor cell and the test hematopoietic cell can be contacted with the agent to be screened prior to contact with the chemotherapeutic agent. For example, the test tumor cell and the test hematopoietic cell can be contacted with the agent to be screened daily for one hour, two hours, three hours, four hours, six hours 12 hours, or one, two, three, four, five, six, or seven days prior to contact with the chemotherapeutic agent. The test tumor cell and the test hematopoietic cell can be contacted with the agent to be screened daily for four days prior to contact with the chemotherapeutic agent, for example.

Also disclosed are methods of treating a subject with cancer, comprising the steps of administering daily to the subject the agent identified by the methods disclosed herein for two, three, four, five, six or seven days prior to administering to the subject an antineoplastic chemotherapeutic agent; and administering to the subject the antineoplastic chemotherapeutic agent. In one embodiment, the agent is administered for four days.

Examples of types of cancer include, but are not limited to, lymphoma (Hodgkins and non-Hodgkins) B-cell lymphoma, T-cell lymphoma, leukemia such as myeloid leukemia and other types of leukemia, mycosis fungoide, carcinoma, adenocarcinoma, sarcoma, glioma, blastoma, neuroblastoma, plasmacytoma, histiocytoma, melanoma, adenoma, hypoxic tumour, myeloma, AIDS-related lymphoma or AIDS-related sarcoma, metastatic cancer, bladder cancer, brain cancer, nervous system cancer, squamous cell carcinoma of the head and neck, neuroblastoma, glioblastoma, ovarian cancer, skin cancer, liver cancer, squamous cell carcinomas of the mouth, throat, larynx, and lung, colon cancer, cervical cancer, breast cancer, cervical carcinoma, epithelial cancer, renal cancer, genitourinary cancer, pulmonary cancer, esophageal carcinoma, head and neck carcinoma, hematopoietic cancer, testicular cancer, colo-rectal cancer, prostatic cancer, and pancreatic cancer.

Also disclosed are methods of screening for an agent that increases the efficacy of an antineoplastic chemotherapeutic agent and reduces the hematotoxicity of the chemotherapeutic agent, comprising administering to a test animal with cancer the chemotherapeutic agent and the agent to be screened; detecting a reduction in cancer cell division or an increase in cancer cell death in the test animal as compared to a control animal with cancer, wherein the control animal is administered the chemotherapeutic agent in the absence of the agent to be screened, a reduction in tumor cell division or an increase in tumor cell death indicating an agent that increases in the efficacy of the chemotherapeutic agent; and detecting an increase in hematopoietic cell division or a reduction in hematopoietic cell death in the test animal as compared to the control animal, an increase in hematopoietic cell division or a decrease in hematopoietic cell death indicating an agent that reduces the hematotoxicity of the chemotherapeutic agent.

The increase in efficacy of the chemotherapeutic agent can comprise an increase in uptake of the chemotherapeutic agent by the cancer cells in the test animal as compared to the cancer cells in the control animal. Furthermore, the reduction in hematotoxicity can comprise a decrease in uptake of the chemotherapeutic agent by the hematopoietic cells in the test animal as compared to the hematopoietic cells of the control animal.

The test animal and control animal can comprise tumor xenografts. The cancer cells can be solid tumor cells. The increase in efficacy of the chemotherapeutic agent can comprise an increase in uptake of the chemotherapeutic agent by the tumor in the test animal as compared to the tumor in the control animal. The reduction in hematotoxicity can comprise a decrease in uptake of the chemotherapeutic agent by the hematopoietic tissues of the test animal as compared to the control animal. Furthermore, the hematopoietic tissue can be selected from either bone marrow or splenic tissue. The test animal can be administered the agent to be screened prior to administration of the chemotherapeutic agent. For example, the test animal can administered the agent to be screened daily for two, three, four, five, six, or seven days prior to administration of the chemotherapeutic agent. In one embodiment, the test animal can be administered the agent to be screened daily for four days prior to administration of the chemotherapeutic agent.

Also disclosed are methods of treating a subject with cancer, comprising the steps of administering daily to the subject the agent described above for three to seven days prior to administering to the subject an antineoplastic chemotherapeutic agent; and administering to the subject the antineoplastic chemotherapeutic agent. In one example, the administration step of (a) for four days.

Also disclosed are agents identified by the methods disclosed herein. Disclosed are embodiments wherein the agent is not dexamethasone or cortisone acetate.

The compositions disclosed herein can also be administered in vivo in a pharmaceutically acceptable carrier. By “pharmaceutically acceptable” is meant a material that is not biologically or otherwise undesirable, i.e., the material may be administered to a subject, along with the nucleic acid or vector, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

The compositions may be administered orally, parenterally (e.g., intravenously), by intramuscular injection, by intraperitoneal injection, transdermally, extracorporeally, topically or the like, including topical intranasal administration or administration by inhalant. Delivery can also be directly to any area of the respiratory system (e.g., lungs) via intubation. The exact amount of the compositions required will vary from subject to subject, depending on the species, age, weight and general condition of the subject, the severity of the allergic disorder being treated, the particular nucleic acid or vector used, its mode of administration and the like. Thus, it is not possible to specify an exact amount for every composition. However, an appropriate amount can be determined by one of ordinary skill in the art using only routine experimentation given the teachings herein.

Parenteral administration of the composition, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein.

The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and McKenzie, Immunolog. Reviews, 129:57-80, (1992); and Roffler, et al., Biochem. Pharmacol, 42:2062-2065, (1991)). Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

The compositions disclosed herein can be used therapeutically in combination with a pharmaceutically acceptable carrier. Suitable carriers and their formulations are described in Remington: The Science and Practice of Pharmacy (19th ed.) ed. A. R. Gennaro, Mack Publishing Company, Easton, Pa. 1995. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. Further carriers include sustained release preparations such as semipermeable matrices of solid hydrophobic polymers, which matrices are in the form of shaped articles, e.g., films, liposomes or microparticles. It will be apparent to those persons skilled in the art that certain carriers may be more preferable depending upon, for instance, the route of administration and concentration of composition being administered.

Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds will be administered according to standard procedures used by those skilled in the art.

Pharmaceutical compositions may include carriers, thickeners, diluents, buffers, preservatives, surface active agents and the like in addition to the molecule of choice. Pharmaceutical compositions may also include one or more active ingredients such as antimicrobial agents, antiinflammatory agents, anesthetics, and the like.

The pharmaceutical composition may be administered in a number of ways depending on whether local or systemic treatment is desired, and on the area to be treated. Administration may be topically (including ophthalmically, vaginally, rectally, intranasally), orally, by inhalation, or parenterally, for example by intravenous drip, subcutaneous, intraperitoneal or intramuscular injection. The disclosed antibodies can be administered intravenously, intraperitoneally, intramuscularly, subcutaneously, intracavity, or transdermally.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulations for topical administration may include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids or binders may be desirable.

Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fimaric acid, or by reaction with an inorganic base such as sodium hydroxide, ammonium hydroxide, potassium hydroxide, and organic bases such as mono-, di-, trialkyl and aryl amines and substituted ethanolamines.

Effective dosages and schedules for administering the compositions may be determined empirically, and making such determinations is within the skill in the art. The dosage ranges for the administration of the compositions are those large enough to produce the desired effect in which the symptoms disorder are effected. The dosage should not be so large as to cause adverse side effects, such as unwanted cross-reactions, anaphylactic reactions, and the like. Generally, the dosage will vary with the age, condition, sex and extent of the disease in the patient, route of administration, or whether other drugs are included in the regimen, and can be determined by one of skill in the art. The dosage can be adjusted by the individual physician in the event of any counterindications. Dosage can vary, and can be administered in one or more dose administrations daily, for one or several days. Guidance can be found in the literature for appropriate dosages for given classes of pharmaceutical products. For example, guidance in selecting appropriate doses for antibodies can be found in the literature on therapeutic uses of antibodies, e.g., Handbook of Monoclonal Antibodies, Ferrone et al., eds., Noges Publications, Park Ridge, N.J., (1985) ch. 22 and pp. 303-357; Smith et al., Antibodies in Human Diagnosis and Therapy, Haber et al., eds., Raven Press, New York (1977) pp. 365-389. A typical daily dosage of the antibody used alone might range from about 1 μg/kg to up to 100 mg/kg of body weight or more per day, depending on the factors mentioned above.

### EXAMPLES

### Example 1

### Dexamethasone as a Chemoprotectant in Cancer Chemotherapy: Hematoprotective Effects and Altered Pharmacokinetics and Tissue Distribution of Carboplatin and Gemcitabine

Hematoprotective strategies can offer new approaches to prevent chemotherapy-induced hematotoxicity. The present study was undertaken to investigate the chemoprotective effects of dexamethasone and its optimal dose and the underlying mechanisms. Lethal toxicity and hematotoxicity of carboplatin were compared in CD-1 mice with or without dexamethasone pretreatment. Plasma and tissue pharmacokinetics of carboplatin were determined in CD-1 mice. Carboplatin was quantified by HPLC. Gemcitabine was analyzed by radioactivity counting. Pretreatment with dexamethasone prevented lethal toxicity of carboplatin in a dose- and schedule-dependent manner. The best protective effects of dexamethasone pretreatment as measured by survival were observed at the dose level of 0.1 mg/mouse/day×5 (80% vs. 10% in controls). Pretreatment with dexamethasone significantly prevented the decrease in granulocyte counts.

To elucidate the mechanisms by which dexamethasone pretreatment reduces hematotoxicity, the effects of dexamethasone pretreatment on pharmacokinetics of carboplatin and gemcitabine were examined in CD-1 mice. Dexamethasone pretreatment significantly decreased carboplatin or gemcitabine uptake in spleen and bone marrow with significant decreases in AUC, T1/2, and Cmax and increase in clearance. Dexamethasone has been shown to significantly decrease host tissue uptake of chemotherapeutic agents, showing a mechanism responsible for chemoprotective effects of dexamethasone.

Abbreviations used are: DEX, dexamethasone; HPLC, high-performance liquid chromatography; PBS, phosphate-buffered saline; AUC, the area under the drug concentration-time curve; Cmax, the maximal concentration; T1/2, elimination half-life; CL, Clearance Introduction.

Materials and Methods

Chemicals, Regents, and Animals

All chemicals and solvents were HPLC grade or of the highest analytical grade available. Methanol, acetonitrile, and acetic acid were purchased from Fisher Chemicals (Atlanta, Ga.). DEX (analytical grade), carboplatin (analytical grade), and triethylamine were purchased from Sigma (St. Louis, Mo.). Perchloric acid was purchased from J. T. Baker Inc. (Phillipsburg, N.J.). Centrifree™ micropartition system (Cat # 4104) was purchased from Millipore Corporation (Bedford, Mass.). Carboplatin (clinical grade) was purchased from Bristol-Myers Squibb Company (Princeton, N.J.) and gemcitabine (clinical grade) was purchased from Eli Lilly and Company (Indianapolis, Ind.). DEX (clinical grade) was purchased from American Regent Laboratories (Shirley, N.J.). [³H]-gemcitabine was obtained from Moravek Biochemicals (Brea, Calif.). Tissue solubilizer (TS-2) was purchased from Research Products Inc (Mt. Prospect, Ill.). The animal use and care protocol was approved by the Institutional Animal Use and Care Committee of the University of Alabama at Birmingham. Male CD-1 mice (4-6 weeks old) were obtained from Charles River Laboratories (Cambridge, Mass.). All animals were fed with commercial diet and water ad libitum for one week prior to the study.

Animal Survival Study

Male CD-1 mice, randomly divided into multiple treatment and control groups (10 mice/group), were given DEX by subcutaneous (s.c.) injection at doses of 0.01, 0.05, 0.1, 0.3 mg/mouse/day or saline (as controls) for 5 days prior to (day −4 to 0) or after (day 0 to 4) a single intraperitoneal (i.p.) injection of carboplatin (600 mg/m² or 200 mg/kg) on day 0. Animals were monitored daily for activity, physical condition, body weight and 14-day survival rates.

Peripheral Blood Cell Counts

sing a protocol similar to the above survival study, the effects of DEX on chemotherapy-induced bone marrow toxicity were studied in male CD-1 mice. DEX (s.c., 0.1 mg/mouse/day for 5 days) was given prior to (day −4-0) or after (day 0 to 4) a single i.p. dose of carboplatin (600 mg/m² or 200 mg/kg). On days −4, 0, 3, 7, 14, peripheral blood samples (60 μL) were obtained from postorbital venous plexus using a microcapillary tube coated with 5% ethylenediaminetetraacetic acid and cell counts were obtained using a Coulter counter. Wright's stained peripheral blood smears were examined by light microscopy and the percentage of lymphocytes, granulocytes and monocytes were recorded.

Pharmacokinetics and Tissue Distribution of Carboplatin

Pharmacokinetic studies were carried out using a protocol similar to that previously described (Wang, 1999) using metabolism cages. Male CD-1 mice were pre-treated with DEX (s.c., 0.1 mg/day/mouse for 5 days) or saline (as controls), and, at 1 hr after the fifth dose of DEX, were given a single intravenously (i.v.) bolus administration of carboplatin (60 mg/kg) via a tail vein. At various times (5, 15, and 30 min, 1, 2, 4, 8, and 24 hr after drug dosing; 3 animals for each time point)), blood samples were collected in heparinized tubes and tissue samples removed. Plasma was separated by centrifugation at 20,000 g for 5 min. Tissues including liver, kidneys and spleen were immediately blotted on Whatman No. 1 filter paper, trimmed of extraneous fat or connective tissue, weighed, and homogenized in 0.9% NaCl physiological saline (5 mL per g of wet tissue weight). The resultant homogenates were stored at −70° C. until further analysis. Bone marrow cells were harvested by flushing the femurs with sterile physiological saline as reported previously (Zhang, 1993). The resultant bone marrow cell suspension was weighed and lysed by sonicating 5 times for periods of 10 sec. Following centrifugation at 20,000 g for 30 min, the supernatant was removed and stored at −70° C. until further analysis.

HPLC Analysis of Carboplatin in Plasma and Tissues

Aboplatin in biological samples was analyzed by an analytical procedure involving micro-filtration and reversed-phase HPLC (Bullen, 1992). 200 μL of plasma or tissue homogenates or bone marrow suspension was added to the reservoir of a Centrifree™ micropartition system. The latter was capped and centrifuged at 2,000 g for 5 min. All filtrates were transferred into a new micro centrifuge tube and 6 μL of the filtrates was injected onto the HPLC column. The HPLC system consisted of a Hewlett Packard 1050 ChemStation with a UV detector (Agilent 1050 series). Determination of Carboplatin was achieved using a LiChrosorb™ diol (10 μm, 250×4.6 mm) analytical column with a LiChroCART 100 RP-18 guard column. The mobile phase for plasma was composed of 98:2 acetonitrile: H₂O (vol/vol) and 89:11 acetonitrile: 0.015% H₃PO₄ (vol/vol) for urine and tissue samples. The flow rates were 2 mL/min (for plasma) and 1.1 mL/min (for urine and tissue samples). The column elute was monitored by UV at 229 nm. Quantitation of plasma or tissue carboplatin was carried out by using an external standard curve (0-4,000.0 μg/mL) that was freshly prepared daily. Linear regression and correlation analysis were carried out to establish the standard peak-area/concentration curves for carboplatin.

Pharmacokinetics and Tissue Distribution of Gemcitabine

Pharmacokinetic studies of gemcitabine were carried out using a protocol similar to carboplatin pharmacokinetics as described above. Male CD-1 mice (3 animals for each time point) were pretreated with DEX as described above, and then given a single i.v. bolus administration of [³H]-gemcitabine (160 mg/kg) via a tail vein. At various times (5, 15, and 30 min, 1, 2, 4, 8, and 24 hr after drug dosing), plasma, bone marrow, and tissues including liver, kidneys, heart, lungs, spleen, brain and tumor were collected and treated as described above.

Quantitation of Gemcitabine by Radioactivity Measurements

The total gemcitabine-derived radioactivity in tissues and body fluids were determined by liquid scintillation spectrometry (LS 6000T A; Beckman, Irvine, Calif.), using a method described previously (Wang, 1999; Zhang, 1995). In brief, plasma samples (50 μL) were mixed with 5 μL of scintillation solvent (Beckman) to determine total radioactivity. Tissue homogenates (50-200 μL) were mixed with 200 μL solubilizer (TS-2) overnight, neutralized with 400 μL of 0.3% acetic acid, and then mixed with scintillation solvent (5 mL) to quantitate the total radioactivity.

Data and Statistical Analysis

The peripheral blood counts were expressed as mean and standard deviations and the significance of differences were analyzed by ANOVA; survival rates in the toxicity study were analyzed by the χ² analysis. The pharmacokinetic parameters of carboplatin were estimated by using WinNonlin programs (Version 2.1, Pharsight, Mountain View, Calif.): the area under the drug concentration-time curve (AUC), the maximal concentration (Cₘₐₓ), the elimination half-life (T_{1/2}), Clearance (CL) and volume of distribution at steady-state (Vₛₛ). The significance of the differences among treated groups and controls were analyzed by ANOVA.

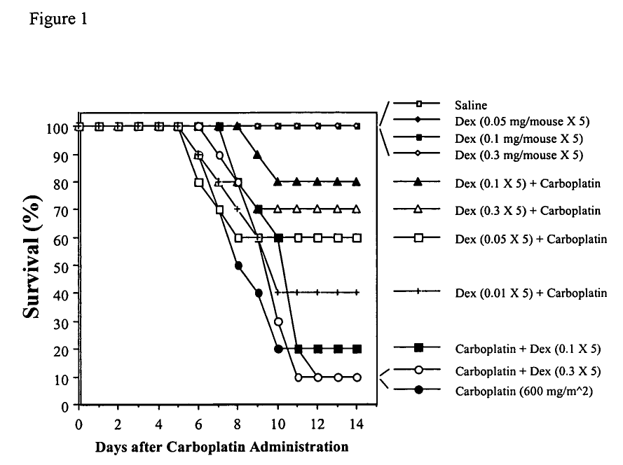

The effect ofpretreatment with DEX on reduction in lethal carboplatin hematotoxicity is schedule and dose dependent. DEX pretreatment significantly reduced mortality of carboplatin in CD-1 mice (FIG. 1). No death occurred in groups treated with DEX (s.c., 0.01-0.3 mg/mouse/day for 5 days) alone or saline. The single i.p. dose of carboplatin alone (600 mg/m² or 200 mg/kg) resulted in 90% mortality rate, which is similar to the previously reported lethal toxicity of carboplatin (Rinehart, 1994, Rinehart 1995). Pretreatment with DEX prevented lethal toxicity of carboplatin in a dose-dependent manner. At a lower dose (0.01 mg/mouse/day for 5 days), DEX pretreatment slightly increased the survival rate (40% vs. 10% in carboplatin controls). The best protective effects as measured by survival were observed at the dose level of 0.1 mg/mouse/day (80% vs. 10% in carboplatin controls). No significant changes in body weights were observed in DEX pre-treated mice compared with the control mice that survived. In the combination treatment with carboplatin and gemcitabine, pretreatment with DEX also reduced the mortality of treated animals.

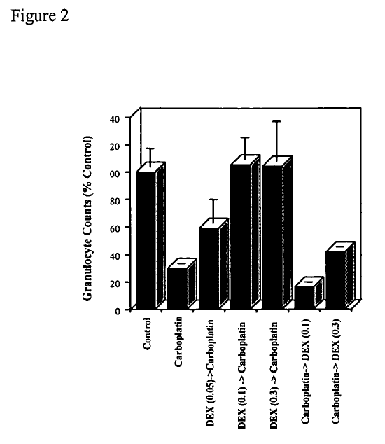

Pretreatment with DEX reduces chemotherapy-induced cytopenias. To examine possible mechanisms responsible for reduced mortality in mice pre-treated with DEX, peripheral blood counts before and after chemotherapy were determined. Mice (10/group) were treated with dexamethasone 0.1 mg/mouse/day s.c. for 5 days and on day 0 received carboplatin (360 mg/m²) alone or in combination with gemcitabine (480 mg/m²) (both i.p., day 0). Peripheral blood counts on days −4, 0, 3, 7, 14 reflect the effect of dexamethasone and demonstrate dexamethasone induction of transient granulocytosis and lymphopenia. Dexamethasone prevented carboplatin or gemcitabine induction of neutropenia. The same experimental design was used to examine the dose-schedule effects of dexamethasone prevention of neutropenia. DEX prevention of neutropenia was dose- and schedule-dependent, the optimal dose was 0.1 mg/kg and postcarboplatin treatment had no effect. As illustrated in FIG. 2, the data showed nadirs of granulocyte counts by different treatment of dexamethasone following carboplatin (single dose, 360 mg/m²) on day 14.

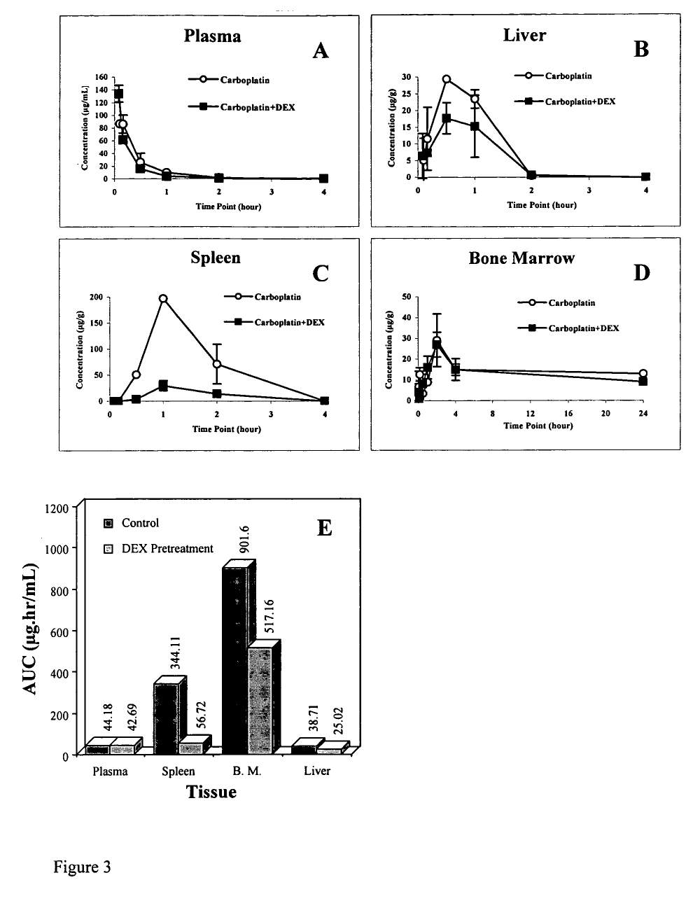

Carboplatin Pharmacokinetics. The carboplatin pharmacokinetic study was performed in CD-1 mice with or without DEX pretreatment. The time-concentration curves are illustrated in FIG. 3. DEX markedly decreased carboplatin concentrations in spleen (p<0.01, FIG. 3C); pharmacokinetic analysis indicated that there were significant decreases in AUC and Cmax and increases in CL in mice pretreated with DEX (p<0.01, FIG. 3E, Table 1). As shown in FIG. 3E, the AUC of carboplatin in spleen from animals pretreated with DEX was approximately 16.5% that of control mice (p<0.01). It was also found that 57.4% carboplatin AUC decrease in bone marrow from animals pretreated with DEX compared with that of control mice (FIGS. 3D and E, Table 1). In addition, pretreatment with DEX decreased carboplatin uptake in liver 40% (FIGS. 3B, 3E, Table 1).

TABLE 1
Pharmacokinetic parameters of carboplatin in CD-1 mice following
pretreatment with DEX or saline. One-compartmental model was fitted to the data of
plasma and tumor time-concentration curves and a first-order absorption, one-
compartmental model was fitted to the data of spleen, bone marrow and liver time-
concentration curves.

| | | | Spleen | | | | Liver | |
|---|---|---|---|---|---|---|---|---|
| | Plasma | | | DEX | Bone Marrow | | | DEX |
| Parameters | DEX (−) | DEX (+) | DEX (−) | (+) | DEX (−) | DEX (+) | DEX (−) | (+) |
| AUC | 44.18 | 42.69 | 344.11 | 56.72 | 901.60 | 517.16 | 38.71 | 25.02 |
| (μg · hr/mL) | | | | | | | | |
| T_{1/2} (hr) | 0.09 | 0.13 | 0.75 | 0.82 | 28.47 | 15.38 | 0.39 | 0.39 |
| Cmax | 352.62 | 227.58 | 118.73 | 17.79 | 20.14 | 20.61 | 25.44 | 16.55 |
| (μg/mL) | | | | | | | | |
| CL | 1.36 | 1.41 | 0.17 | 1.06 | 0.07 | 0.12 | 1.55 | 2.40 |
| (mL/g/hr) | | | | | | | | |
| Vss (mL/g) | 0.17 | 0.26 | 0.19 | 1.25 | 2.75 | 2.57 | 0.87 | 1.35 |

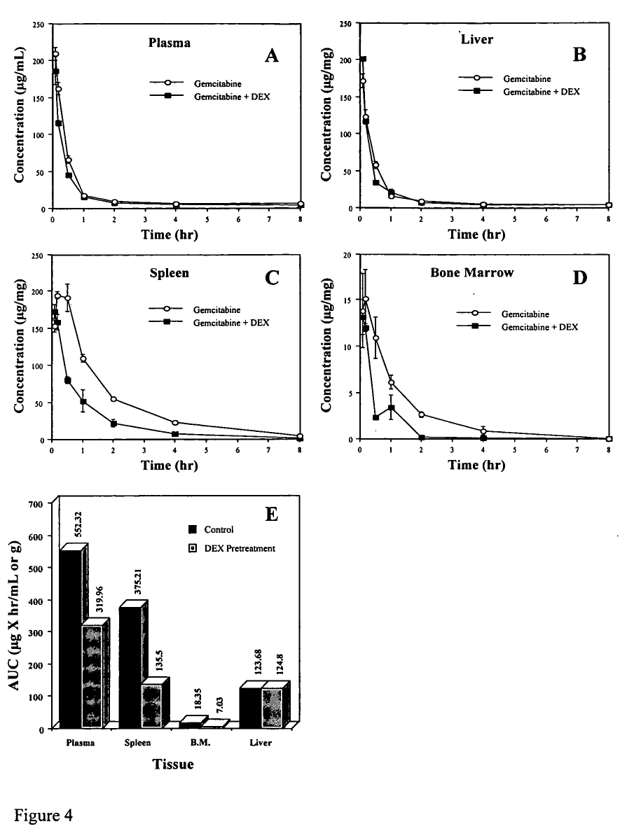

Gemcitabine Pharmacokinetics. The gemcitabine pharmacokinetic study was also carried out in CD-1 mice using a similar protocol as previously described. The time-concentration curves are illustrated in FIG. 4. Slight but significant differences in plasma pharmacokinetics of gemcitabine were observed between control and mice pre-treated with DEX (FIG. 4A). Pharmacokinetic analysis indicated that plasma AUC was decreased with DEX pretreatment (FIG. 4E, Table 1). No significant differences in liver drug concentrations were found between control and mice pre-treated with DEX (FIG. 4B). However, DEX markedly decreased gemcitabine concentrations in spleen and bone marrow (p<0.01, FIG. 4C, D); pharmacokinetic analysis indicated that there were significant decreases in AUC and Cmax and increases in the CL in mice pre-treated with DEX (p<0.01, FIG. 3E, Table 2). The AUCs of gemcitabine in spleen and bone marrow from animals pretreated with DEX were approximately 36% and 38% that of control mice, respectively (p<0.01, FIG. 4E, Table 2).

TABLE 2
Pharmacokinetic parameters of gemcitabine in CD-1 mice following
pretreatment with DEX or saline.

| | | | | | Bone Marrow | | | |
|---|---|---|---|---|---|---|---|---|
| | Plasma | | Spleen | | | DEX | Liver | |
| Parameters | DEX (−) | DEX (+) | DEX (−) | DEX (+) | DEX (−) | (+) | DEX (−) | DEX (+) |
| AUC | 552.32 | 319.96 | 375.21 | 135.50 | 18.35 | 7.03 | 123.68 | 124.80 |
| (μg · hr/mL) | | | | | | | | |
| T_{1/2} (hr) | 1.44 | 0.87 | 1.32 | 0.49 | 0.79 | 0.31 | 0.54 | 0.52 |
| Cmax | 264.13 | 253.68 | 196.84 | 193.29 | 16.12 | 15.82 | 158.43 | 166.94 |
| (μg/mL) | | | | | | | | |
| CL | 0.29 | 0.50 | 0.43 | 1.18 | 8.72 | 22.76 | 1.29 | 1.28 |
| (mL/g/hr) | | | | | | | | |
| Vss (mL/g) | 16.64 | 10.81 | 0.81 | 0.83 | 9.93 | 10.12 | 1.01 | 0.96 |

### Example 2

### Pretreatment with Dexamethasone Increases Antitumor Activity of Carboplatin and Gemcitabine in Mice Bearing Human Cancer Xenografts: In Vivo Activity, Pharmacokinetics and Clinical Implications for Cancer Chemotherapy

Since dexamethasone (DEX) decreases tumor interstitial fluid pressure (TIFP) and can increase drug uptake in tumor tissues, pretreatment with DEX can enhance the antitumor activity of cancer chemotherapeutic agents. Antitumor activities of carboplatin and gemcitabine with or without DEX pretreatment were determined in six murine-human cancer xenograft models, including cancers of colon (LS174T), lung (A549 and H1299), and breast (MCF-7 and MDA-MB-468) and glioma (U87MG). Effects of DEX on plasma and tissue pharmacokinetics of carboplatin and gemcitabine were also determined by using the LS174T, A549 and H1299 models. DEX pretreatment significantly increased the efficacy of carboplatin, gemcitabine or combination of both drugs by 2-4-fold in all xenograft models tested. Without DEX treatment, the tumor exposure to carboplatin, measured by the area under the curve (AUC), was markedly lower than normal tissues (4% of spleen, 3% of bone marrow and 23% of liver AUCs). However, DEX pretreatment significantly increased tumor carboplatin uptake, including 200% increase in AUC, 100% increase in maximum concentration, and 160% decrease in Clearance. DEX pretreatment similarly increased gemcitabine uptake in tumors.

### MATERIALS AND METHODS

Chemicals and Reagents. All chemicals and solvents were HPLC grade or of the highest analytical grade available. Methanol, acetonitrile, and acetic acid were purchased from Fisher Chemicals (Atlanta, Ga.). DEX (analytical grade), carboplatin (analytical grade), and triethylamine were purchased from Sigma (St. Louis, Mo.). Perchloric acid was purchased from J. T. Baker Inc. (Phillipsburg, N.J.). Centrifree™ micropartition system (Cat. # 4104) was purchased from Millipore Corporation (Bedford, Mass.). Cell culture media, fetal bovine serum (FBS); phosphate-buffered saline (PBS), sodium pyruvate, non-essential amino acids, penicillin-streptomycin and other cell culture supplies were provided by the Comprehensive Cancer Center Media Preparation Shared Facility, University of Alabama at Birmingham. Carboplatin (clinical grade) was purchased from Bristol-Myers Squibb Company (Princeton, N.J.) and gemcitabine (clinical grade) was purchased from Eli Lilly and Company (Indianapolis, Ind.). DEX (clinical grade) was purchased from American Regent Laboratories (Shirley, N.J.). Matrigel basement membrane matrix was obtained from Becton Dickinson Labware (Bedford, Mass.). [³H]-gemcitabine was obtained from Moravek Biochemicals (Brea, Calif.). Tissue solubilizer (TS-2) was purchased from Research Products Inc (Mt. Prospect, Ill.).

Animals. Female athymic nude mice (nu/nu, 4-6 weeks) were obtained from Frederick Cancer Research Facility (Frederick, MD). All animals were fed with commercial diet and water ad libitum for one week prior to the study.

Cell Culture. The cell lines of human cancers (colon LS174T, breast MCF-7 and MDA-MB-468, and lung A549) and glioma (U87-MG) were obtained from American Type Culture Collection (Rockville, Md.) and cultured according to their instruction. LS174T cells were cultured in modified Earle's medium with 0.1 mM nonessential amino acids and Earle's balanced salt solution containing 10% FBS. MCF-7 cells were grown in modified Earle's medium containing 10% FBS, 1 mM non-essential amino acids and Earle's balanced salt solution, 1 mM sodium pyruvate and 10 mg/L bovine insulin. MDA-MB-468 cells were grown in Dulbecco's modified Eagle's/F-12 Ham's medium (1:1 mixture) containing 10% FBS. U87-MG cells were cultured in Eagle minimal essential medium supplemented with 10% FBS, 1% sodium pyruvate and 1% non-essential amino acids. A549 cells were cultured in Ham's F-12K medium containing 10% FBS. The lung cancer cell line H1299 was kindly provided by Dr. J. Chen (Moffit Cancer Center, Tampa, Fla.) and was grown in Dulbecco's modified Eagle's medium containing 10% FBS. All media contained 1% penicillin-streptomycin.

Animal Tumor Models. Human cancer xenograft models were established using the methods reported previously (26-29). When confluence reached 80%, cultured LS174T, MCF-7, MDA-MB-468, U87-MG, A549 and H1299 cells were harvested from the monolayer cultures, washed with the above indicated serum-free medium and resuspended in the same medium with Matrigel® basement membrane matrix at a 3:1 ratio, and then injected subcutaneously (s.c.) (5×10⁶ cells, total volume 0.2 mL) into the left inguinal area of the nude mice. The animals were monitored for activity, physical condition, determination of body weight, and measurement of tumor growth. Tumor growth was determined by caliper measurement in two perpendicular diameters of the implant every other day. Tumor weight (in g) was calculated by the formula, 1/2a×b² where “a” is the long diameter and “b” is the short diameter (in cm).

In vivo Chemotherapy. Nude mice bearing human cancer xenografts were randomly divided into various treatment and control groups (5 mice/group). In treatment groups, animals were pre-treated with DEX subcutaneously (s.c., 0.1 mg/day for 5 days, Day −4-0) or saline (as controls). Carboplatin was administered intraperitoneally (i.p.) at a single dose of 120 mg/kg (360 mg/m²) on day 0. Gemcitabine was given at a single i.p. dose of 160 mg/kg (480 mg/m²) for U87-MG model and two doses of 160 mg/kg (480 mg/m²) for other models.

Pharmacokinetics and Tissue Distribution of Carboplatin. Pharmacokinetic studies were carried out using a protocol similar to that previously described (26) using metabolism cages. Female nude mice bearing human colon cancer LS174T xenografts or human lung cancer A549 xenografts were used (3 animals for each time point). Animals were pre-treated with DEX (s.c., 0.1 mg/day/mouse for 5 days) or saline (as controls) and, at 1 hr after the fifth dose of DEX, were given a single intravenously (i.v.) bolus administration of carboplatin (60 mg/kg) via a tail vein. At various times (5, 15, and 30 min, 1, 2, 4, 8, and 24 hr after carboplatin dosing), blood samples were collected in heparinized tubes and tissue samples removed. Plasma was separated by centrifugation at 20,000 g for 5 min. Tissues including liver, kidneys, spleen, and tumor were taken at various times and immediately blotted on Whatman No. 1 filter paper, trimmed of extraneous fat or connective tissue, weighed, and homogenized in 0.9% NaCl physiological saline (5 mL per gram of wet weight). The resultant homogenates were stored at −70° C. until further analysis. Bone marrow cells were harvested by flushing the femurs with sterile physiological saline as reported previously (30). Briefly, after removing the distal and proximal ends of femur, each end of the bone was punctured with a 20-gauge needle fitted with a 5 mL syringe containing 1 mL of sterile physiological saline (pre-weighed). The bone was then held with a forceps over a test tube and the needle was inserted into the hole in the proximal end. The 1 mL saline was flushed through the bone into the tube by applying gentle, steady pressure to the plunger. The syringe was then filled with 5 ml of air that was gently forced through the bone to remove the remaining saline and bone marrow in the shaft. The resultant bone marrow cell suspension was weighed and lysed by sonicating 5 times for periods of 10 sec. Following centrifugation at 20,000 g for 30 min, the supernatant was removed and stored at −70° C. until further analysis.

HPLC Analysis of Carboplatin in Plasma and Tissues. Carboplatin in biological samples was analyzed by an analytical procedure involving micro-filtration and reversed-phase HPLC (31). 200 μL of plasma or tissue homogenates or bone marrow suspension was added to the reservoir of a Centrifree™ micropartition system. The latter was capped and centrifuged at 2,000 g for 5 min. All filtrates were transferred into a new micro centrifuge tube and 6 μL of the filtrates was injected onto the HPLC column. The HPLC system consisted of a Hewlett Packard 1050 ChemStation with a UV detector (Agilent 1050 series). Determination of Carboplatin was achieved using a LiChrosorb™ diol (10 μm, 250×4.6 mm) analytical column with a LiChroCART 100 RP-18 guard column. The mobile phase for plasma was composed of 98:2 acetonitrile:H₂O (vol/vol) and 89:11 acetonitrile: 0.015% H₃PO₄ (vol/vol) for urine and tissue samples. The flow rates were 2 mL/min (for plasma) and 1.1 mL/min (for urine and tissue samples). The column elute was monitored by UV at 229 nm. Quantitation of plasma or tissue carboplatin was carried out by using an external standard curve (0-4,000.0 μg/mL) that was freshly prepared on a daily basis. Linear regression and correlation analysis were carried out to establish the standard peak-area/concentration curves for carboplatin.

Pharmacokinetics and Tissue Distribution of Gemcitabine. Female nude mice bearing human lung cancer H1299 xenograft were used (3 mice for each time point). Animals were pre-treated with DEX (s.c., 0.1 mg/day/mouse for 5 days) or saline (as controls) and, at 1 hr after the fifth dose of DEX, were given a single intravenously (i.v.) bolus administration of [³H]-gemcitabine (160 mg/kg) via a tail vein. At various times (5, 15, and 30 min, 1, 2, 4, 8, and 24 hr after drug dosing), blood and tissues including liver, kidneys, heart, lungs, spleen, brain and tumor were collected. Plasma was separated by centrifugation, and tissue samples were immediately weighed, and homogenized in 0.9% NaCl physiological saline (5 mL per g of wet weight). Bone marrow cells were harvested by using the same method described above. Gemcitabine concentrations in biological samples were analyzed by radioactivity counting.

Quantitation of Gemcitabine by Radioactivity Measurements. The total radioactivities of gemcitabine in tissues and body fluids were determined by liquid scintillation spectrometry (LS 6000T A; Beckman, Irvine, Calif.), using a method described previously. In brief, plasma samples (50 μL) were mixed with 5 mL of scintillation solvent (Beckman) to determine total radioactivity. Tissue homogenates (50-200 μL) were mixed with 200 μL solubilizer (TS-2) overnight, neutralized with 400 μL of 0.3% acetic acid, and then mixed with scintillation solvent (5 mL) to permit quantitation of total radioactivity.

Histology and Immunohistochemistry of A549 Xenografts. A549 xenografts from treated nude mice were removed on Day 35 and fixed and stained with hematoxylin-eosin (H/E) or snap frozen in liquid N₂. Immunohistochemical staining was undertaken with rat anti-mouse CD45 (leukocyte common antigen, Ly-5; PharMingen, BD Biosciences) using a Daka Immunohistochemistry kit on frozen tumor tissue. Controls, including anti-mouse CD45 without second antibody and second antibody alone, were negative in the control CD-1 mouse spleens.

Data and Statistical Analysis. The antitumor activity (tumor mass) and peripheral blood counts were expressed as mean and standard deviations and the significance of differences were analyzed by ANOVA. The pharmacokinetic parameters of carboplatin and gemcitabine were estimated by using WinNonlin programs (Version 2.1, Pharsight, Mountain View, Calif.): the area under the drug concentration-time curve (AUC), the maximal concentration (Cₘₐₓ), the elimination half-life (T_{1/2}), Clearance (CL), and volume of distribution at steady-state (Vₛₛ).

### RESULTS

Pretreatment with DEX Enhances Antitumor Activity of Carboplatin and Gemcitabine In Vivo.

The effects of DEX pretreatment on antitumor activity of carboplatin and/or gemcitabine were studied in six murine human cancer xenograft models. When mean tumor mass reached 44-68 mg, animals were treated with s.c. DEX at a dose of 0.1 mg/day for 5 days, followed by chemotherapy.

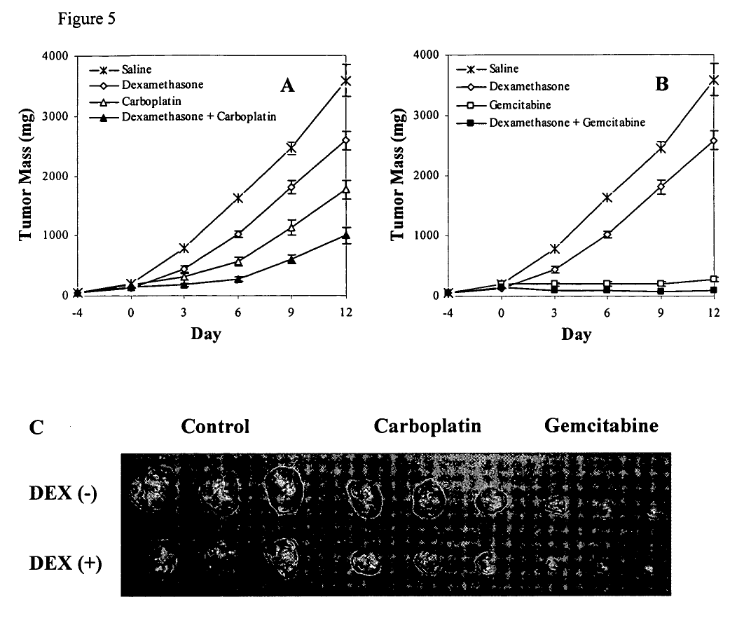

Colon Cancer LS174T Model. As illustrated in FIG. 5, the effect of DEX pretreatment on carboplatin or gemcitabine antitumor activity was demonstrated in nude mice bearing human colon cancer LS174T (p53 mutant) xenografts. DEX alone (s. c., 0.1 mg/day for 5 days, days −4 to 0) showed slight inhibitory effects on tumor growth. Carboplatin was given ip on day 0, at a clinically relevant dose (120 mg/kg or 360 mg/m²). Pretreatment with DEX markedly increased the therapeutic effect of carboplatin by approximately 80% (P<0.01, FIG. 5A). DEX pretreatment also increased anticancer effects of gemcitabine (FIG. 5B). Representative xenograft tumors removed from various treatment groups are shown in FIG. 5C.

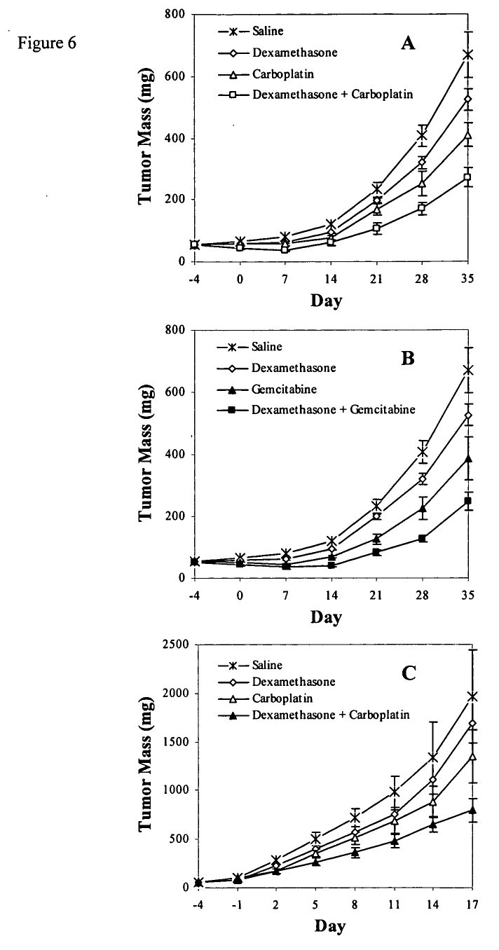

Lung Cancer Models. The effects of DEX pretreatment on carboplatin chemotherapy were further investigated in human lung cancer A549 (p53 mutant) and H1299 (p53 null) models. In the A549 model, pretreatment with DEX significantly increased the therapeutic effectiveness of carboplatin by 50% (P<0.05, FIG. 6A) and gemcitabine by 60% (P<0.05, FIG. 6B). In the H1299 model, DEX pretreatment significantly also increased anticancer effects of carboplatin by 70% (P<0.05, FIG. 6C).

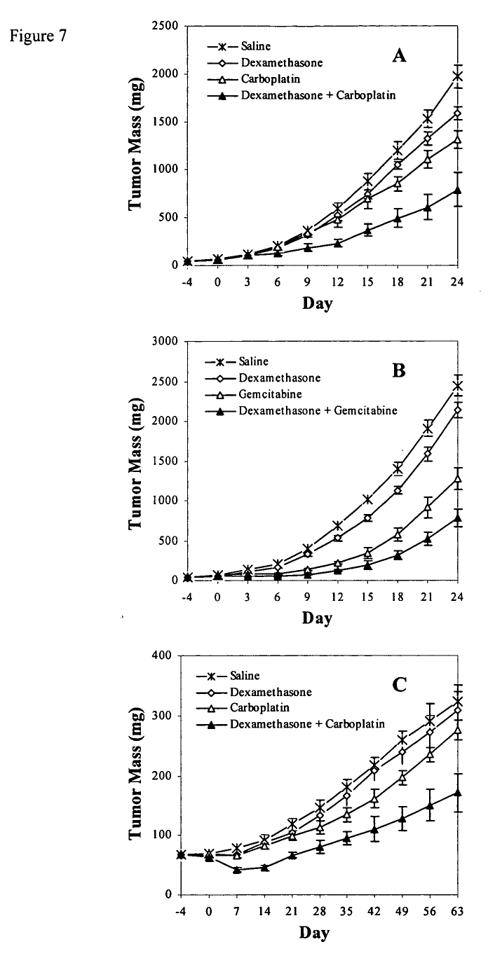

Breast Cancer Models. In two human breast cancer models MCF-7 (p53 wild type) and MDA-MB-468 (p53 mutant), similar effects of DEX pretreatment on carboplatin and gemcitabine chemotherapy were demonstrated (FIG. 7). In the MCF-7 model, both DEX and carboplatin alone had limited effects on tumor growth. However, pretreatment with DEX significantly increased the efficacy of carboplatin (by 66%, P<0.05, FIG. 7A). Pretreatment with DEX also increased the efficacy of gemcitabine (by 60%, P<0.05, FIG. 7B). In MDA-MB-468 model, DEX or carboplatin alone had no effects on tumor growth, but pretreatment with DEX followed by carboplatin significantly inhibited tumor growth (P<0.05, FIG. 6C).

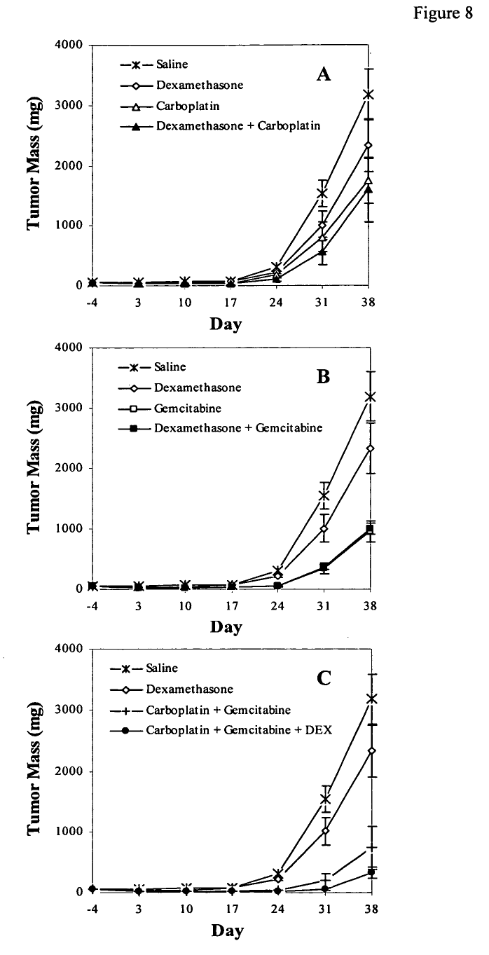

Glioma Model Pretreatment with DEX had no remarkable effects on the therapeutic effectiveness of carboplatin (FIG. 8A) or gemcitabine therapy (FIG. 8B) alone in the human glioma model U87MG (p53 wild type), but significantly increased therapeutic effectiveness of combination therapy of carboplatin and gemcitabine (by 130%, p<0.05, FIG. 8C).

Toxicity of Carboplatin, Gemcitabine and DEX. In the aforementioned human cancer models, DEX showed minimal effects on tumor growth. Effective, clinically relevant doses of carboplatin and gemcitabine were chosen which would not induce mortality or morbidity for these studies. As expected, no death occurred. By inspection and measuring animal weights, carboplatin and gemcitabine were tolerated well. In addition, DEX pretreatment had no effect on toxicity profiles of the two chemotherapeutic agents.

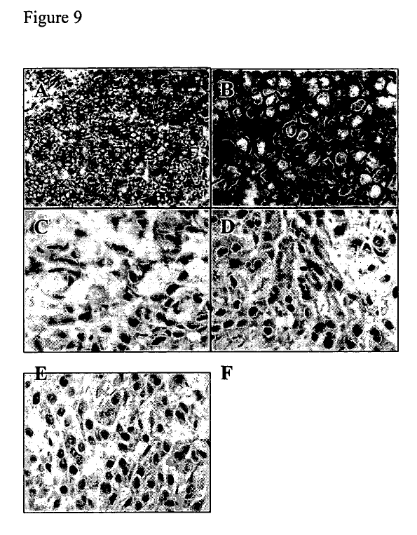

Histology of A549 xenografts. Hematoxylin-eosin (H/E) stains of all four tumors demonstrated similar areas of necrosis, fibrosis and vascular development (data not shown). Rare inflammatory cells were observed. This was confirmed with CD45 immunohistochemistry staining which demonstrated very infrequent CD45 cells (FIG. 9). There were no differences in CD45 staining among treatment groups, showing that the effects of DEX on antitumor effects of chemotherapeutic agents as observed above are not related to DEX reduction in the number of infiltrating CD45 cells.

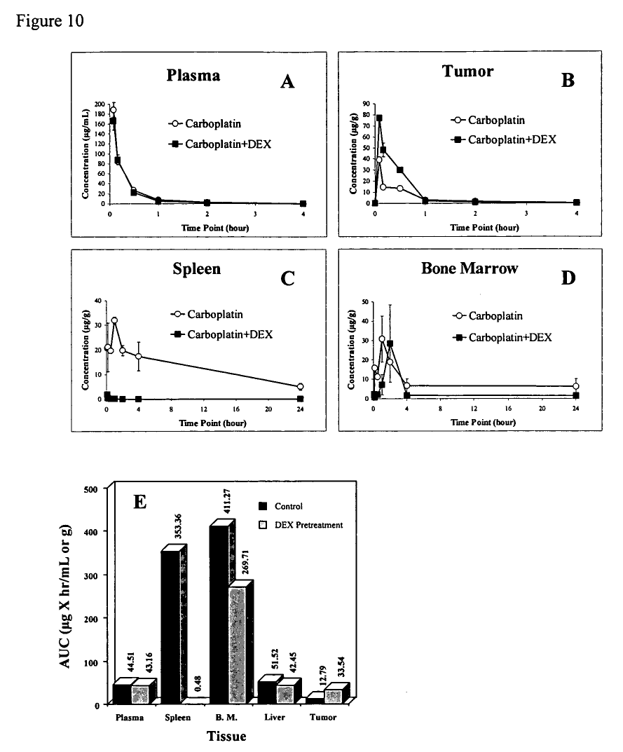

Nude Mice Bearing Human Colon Cancer LS174T Xenografts. The first carboplatin pharmacokinetic study was performed in nude mice bearing LS174T xenografts (500-1000 mg tumor mass). The time-concentration curves are illustrated in FIG. 10. No significant differences in plasma pharmacokinetics of carboplatin were observed between control and mice pre-treated with DEX (FIG. 10A). However, DEX significantly increased tumor carboplatin concentrations (FIG. 10B). Pharmacokinetic analysis indicated that there were significant increases in tumor AUC and Cmax and decreases in the CL in mice pre-treated with DEX (p<0.05, Table 3A). In contrast, pretreatment with DEX decreased carboplatin uptake in spleen (FIG. 10C). Pharmacokinetic analysis indicated that there were significant decreases in splenic AUC, T_{1/2}, and Cmax and an increases in CL in mice pre-treated with DEX (p<0.05, Table 3A). Decreases in bone marrow carboplatin concentrations were also observed (FIG. 10D, Table 3A). No significant differences in liver carboplatin concentrations were found between control and mice pre-treated with DEX. As shown in FIG. 10E, without DEX treatment, the tumor exposure to carboplatin, measured by AUC, was markedly lower than normal tissues (4% of spleen, 3% of bone barrow, and 23% of liver AUCs). However, DEX significantly increased tumor carboplatin uptake, including 200% increase in AUC, 100% increase in Cmax, and 160% decrease in Clearance. (p<0.05).

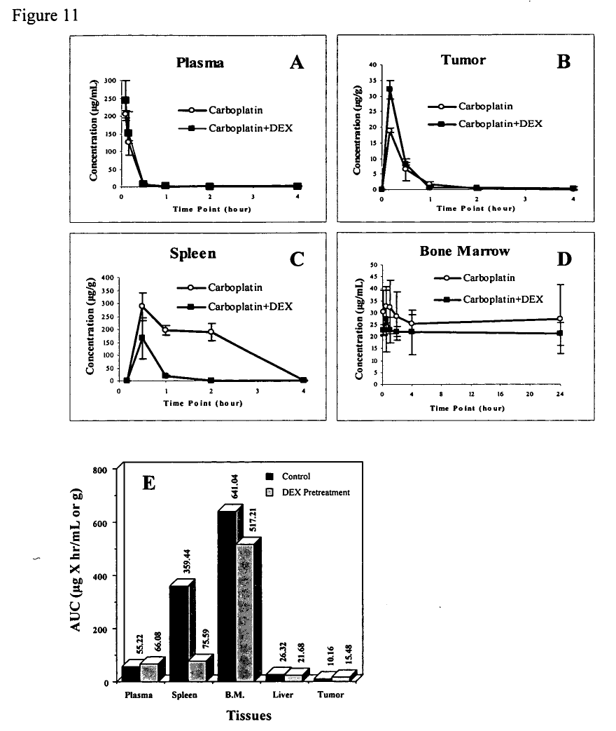

Nude Mice Bearing Human Lung Cancer A549 Xenografts. Carboplatin pharmacokinetic studies were further performed in nude mice bearing human lung cancer A549 xenografts (approximately 500 mg tumor mass). The time-concentration curves are illustrated in FIG. 11. No significant differences in plasma pharmacokinetics of carboplatin were observed between control and mice pre-treated with DEX (FIG. 11A, Table 3B). However, DEX significantly increased tumor carboplatin concentrations (FIG. 11B). Pharmacokinetic analysis indicated that there was a 50% increase in tumor AUC in mice pre-treated with DEX (FIG. 11E, Table 3B). In contrast, pretreatment with DEX decreased carboplatin uptake in spleen (FIG. 11C). Pharmacokinetic analysis indicated that there were significant decreases in AUC, T_{1/2}, and Cmax and an increase in CL in mice pre-treated with DEX (p<0.05, Table 3B, FIG. 11E). Decreases in bone marrow carboplatin concentrations were also observed (FIG. 11D, Table 3B).

TABLE 3A
Pharmacokinetic parameters of carboplatin in nude mice bearing LS174T xenografts
following pretreatment with DEX or saline.
One-compartmental model was fitted to the data of plasma and tumor time-concentration curves.

| | Plasma | | Spleen | | Bone Marrow | | Liver | | Tumor | |
|---|---|---|---|---|---|---|---|---|---|---|
| Parameters | DEX (−) | DEX (+) | DEX (−) | DEX (+) | DEX (−) | DEX (+) | DEX (−) | DEX (+) | DEX (−) | DEX (+) |
| AUC | 44.51 | 43.16 | 353.36 | 0.48 | 411.27 | 269.71 | 51.52 | 42.45 | 12.79 | 33.54 |
| (μg · hr/mL) | | | | | | | | | | |
| T_{1/2} (hr) | 0.08 | 0.11 | 9.91 | 0.04 | 11.16 | 6.84 | 0.49 | 0.56 | 0.20 | 0.26 |
| Cmax | 375.27 | 280.80 | 24.20 | 9.44 | 23.36 | 22.56 | 26.97 | 19.41 | 44.16 | 89.80 |
| (μg/mL) | | | | | | | | | | |
| CL | 1.35 | 1.39 | 0.17 | 126.35 | 0.15 | 0.22 | 1.16 | 1.41 | 4.69 | 1.79 |
| (mL/g/hr) | | | | | | | | | | |
| Vss (mL/g) | 0.16 | 0.21 | 2.43 | 6.36 | 2.42 | 2.17 | 0.82 | 1.14 | 1.36 | 0.67 |

TABLE 3B
Pharmacokinetic parameters of carboplatin in nude mice bearing A549 xenografts following
pretreatment with DEX or saline.

| | Plasma | | Spleen | | Bone Marrow | | Liver | | Tumor | |
|---|---|---|---|---|---|---|---|---|---|---|
| Parameters | DEX (−) | DEX (+) | DEX (−) | DEX (+) | DEX (−) | DEX (+) | DEX (−) | DEX (+) | DEX (−) | DEX (+) |
| AUC | 55.22 | 66.08 | 359.44 | 75.59 | 641.04 | 517.21 | 26.32 | 21.68 | 10.16 | 15.48 |
| (μg · hr/mL) | | | | | | | | | | |
| T_{1/2} (hr) | 0.11 | 0.11 | 0.73 | 0.11 | 0.50 | 0.50 | 1.00 | 1.00 | 0.22 | 0.17 |
| Cmax | 357.34 | 411.89 | 341.99 | 482.14 | 32.58 | 27.29 | 7.00 | 5.91 | 32.15 | 64.09 |
| (μg/mL) | | | | | | | | | | |
| CL | 1.09 | 0.91 | 0.17 | 0.79 | 0.01 | 0.01 | 3.99 | 4.79 | 5.90 | 3.88 |
| (mL/g/hr) | | | | | | | | | | |
| Vss (mL/g) | 0.17 | 0.15 | 0.18 | 0.12 | 1.97 | 2.72 | 8.31 | 9.05 | 1.87 | 0.94 |

DEX Alters Pharmacokinetics of Gemcitabine Chemotherapy in Murine-Human Cancer Xenograft Models.

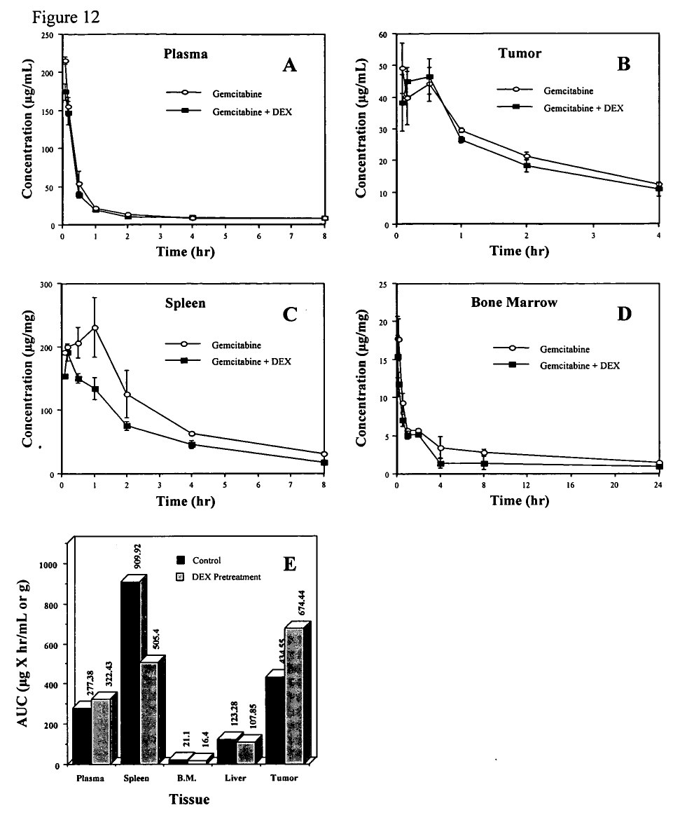

The gemcitabine pharmacokinetic study was carried in nude mice bearing human lung cancer H1299 xenografts (approximately 500 mg tumor mass). The time-concentration curves are illustrated in FIG. 12. No significant differences in plasma pharmacokinetics of gemcitabine were observed between control and mice pre-treated with DEX (FIG. 12A). However, DEX pretreatment increased tumor gemcitabine concentrations (FIG. 12B). Pharmacokinetic analysis indicated that there was a 60% increase in tumor AUC in mice pre-treated with DEX (Table 4). In contrast, pretreatment with DEX decreased gemcitabine uptake in spleen (FIG. 12C). Pharmacokinetic analysis indicated that there were significant decreases in splenic AUC, T_{1/2}, and Cmax and an increase in Clearance in mice pre-treated with DEX (p<0.05, Table 4, FIG. 12E). A slight decrease in bone marrow gemcitabine concentration was also observed (FIG. 12D).

TABLE 4
Pharmacokinetic parameters of gemcitabine in nude mice bearing H1299 xenografts
following pretreatment with DEX or saline.

| | Plasma | | Spleen | | Bone Marrow | | Liver | | Tumor | |
|---|---|---|---|---|---|---|---|---|---|---|
| Parameters | DEX (−) | DEX (+) | DEX (−) | DEX (+) | DEX (−) | DEX (+) | DEX (−) | DEX (+) | DEX (−) | DEX (+) |
| AUC | 277.38 | 322.43 | 909.92 | 505.40 | 21.10 | 16.41 | 123.27 | 107.85 | 434.55 | 674.44 |
| (μg · hr/mL) | | | | | | | | | | |
| T_{1/2} (hr) | 0.67 | 0.94 | 2.84 | 1.95 | 0.79 | 0.79 | 0.50 | 0.47 | 6.12 | 10.02 |
| Cmax | 286.72 | 237.16 | 221.99 | 179.42 | 18.53 | 14.36 | 171.80 | 157.46 | 49.15 | 46.66 |
| (μg/mL) | | | | | | | | | | |
| CL | 0.58 | 0.50 | 0.18 | 0.32 | 7.58 | 9.75 | 1.30 | 1.48 | 0.37 | 0.23 |
| (mL/g/hr) | | | | | | | | | | |
| Vss (mL/g) | 5.96 | 9.90 | 0.72 | 0.89 | 8.64 | 11.14 | 0.93 | 1.02 | 9.01 | 12.72 |

### Example 3

### In Vivo Results

It has been demonstrated that the macrophage driven primary immune response to epithelial cell cancers and resultant cytokine release alters tumor physiology by contributing to neo-angiogenesis, increasing tumor interstitial fluid pressure and volume. Further this process alters normal vasculature by inducing systemic leaky capillary physiology. Intensive anti-inflammatory therapy immediately pre-chemotherapy reverses this process and increases efficacy and decreases toxicity of chemotherapy. Preclinical murine studies demonstrated that administration of Dex prior to chemotherapy, decreases CG toxicity and AUC of CG in bone marrow and spleen and increases AUC of CG in tumor and CG anti-tumor effects (Clin. Can. Res. 10:1633, 2004 and Can. Chemo. Pharm. 53:459, 2004) and decreases systemic and intra-tumor cytokine release from macrophages. Therefore the following clinical trial was undertaken.

Untreated, stage 4, NSCLC patients were treated with 4 courses of C, AUC 5.5 day 1, and G 1 g/m² days 1 and 8 q21 days. Patients were randomized at a 1:2:2 ratio to receive no Dex (Cohort 1), or Dex 8 mg bid (Cohort 2) or 16 mg bid (Cohort 3), 4 days prior to and the day of each chemotherapy treatment. Dex was given only in courses 2, 3, and 4 so that toxicities between course 1 vs. course 2 comparisons could be made.

Patients accrued: 28 of 30 planned patients are accrued; 23 patients who have received at least 2 courses are reported here; 5 patients received only one course due to neutropenia/death (1), hepatotoxicity (1), neurotoxicity (1), withdrew consent (1), and too early (1). Demographics: m:f, 11: 12; median PS-1 (ECOG), and age-65 years. Non-hematologic toxicity was similar in cohorts 1, 2 and 3 and in courses 1 and 2. No Dex DLTs were observed. Hematologic toxicity: Without Dex (Cohort 1) nadirs and other hematologic toxicity parameters worsened after course 1 but improved with Dex (Cohorts 2&3). For example, the ratio of nadir counts (course2÷coursel) demonstrated. improvement with Dex: AGC nadir ratios for Cohorts 1, 2, and 3 (M±SE): 0.99±02., 2.68±0.8 (p<0.03, Cohorts 1 vs. 2) and 2.94±0.7 (p<0.03, Cohorts 1 vs. 3); platelet nadir ratios for cohorts 1, 2, and 3: 0.81±0.0.2, 2.68±0.6 (p<0.04), and 2.72±0.6 (p<0.03). Other parameters, including recovery times, neutropenic days, and number of courses given, demonstrated similar trends when comparisons were made between courses 1 & 2 or between cohorts. Data trends also showed that 16 mg was effective in reducing toxicity. Responses were more frequent in Cohorts 2 & 3 (with Dex): Cohort 1 (n=5): 2 PR, 2 PD, 1 SD; Cohort 2 (n=8): 7 PR, 1 PD; Cohort 3 (n=10): 8 PR, 1 PD, and 1 SD.

Throughout this application, various publications are referenced. The disclosures of these publications in their entireties are hereby incorporated by reference into this application in order to more fully describe the state of the art to which this invention pertains.

### REFERENCES

Aglietta, M., Monzeglio, C., and Pasquino, P. Short-term administration of granulocyte-macrophage colony stimulating factor decreases hematopoietic toxicity of cytostatic drugs. Cancer, 72: 2970-2973, 1993.

Aman, M. J., Keller, U., and Derigs, G. Regulation of cytokine expression by interferon-α in human bone marrow stromal cells: inhibition of hematopoietic growth factors and induction of interleukin-a receptor antagonist. Blood, 84: 4142-4150, 1994.

Auphan, N., DiDonato, J. A., Rosette, C., Helmberg, A. and Karin, M. Immunosuppression by glucocorticoids: inhibition of NF-kappa B activity through induction of I kappa B synthesis. Science, 270: 286-290, 1995.

Barbera-Guillem, E., Nyhus, J. K., Wolford, C. C., Friece, C. R. and Sampsel, J. W. Vascular endothelial growth factor secretion by tumor-infiltrating macrophages essentially supports tumor angiogenesis, and IgG immune complexes potentiate the process. Cancer Res., 62: 7042-7049, 2002.

Bernardi, R. J., Trump, D. L., Yu, W. D., McGuire, T. F., Hershberger, P. A., and Johnson, C. S. Combination of 1alpha, 25-dihydroxyvitamin D (3) with dexamethasone enhances cell cycle arrest and apoptosis: role of nuclear receptor cross-talk and Erk/Akt signaling. Clin. Cancer Res., 7: 4164-4173, 2001.

Berrebi, D., Bruscoli, S., Cohen, N., Foussat, A., Migliorati, G., Bouchet-Delbos, L., Maillot, M. C., Portier, A., Couderc, J., Galanaud, P., Peuchmaur, M., Riccardi, C. and Emilie, D. Synthesis of glucocorticoid-induced leucine zipper (GILZ) by macrophages: an anti-inflammatory and immunosuppressive mechanism shared by glucocorticoids and IL-10. Blood, 101: 729-738, 2003.

Betticher, D., Anderson, H., and Ranson, M. Carboplatin combined with amifostine, a bone marrow protectant, in the treatment of non-small-cell lung cancer: a randomized phase II study. Br. J. Cancer, 72: 1551-1555, 1995.

Braunschweiger, P. G. and Schiffer, L. M. Effect of dexamethasone on vascular finction in RIF-1 tumors. Cancer Res., 46: 3299-3303, 1986.

Braunschweiger, P. G., Ting, H. L., and Schiffer, L. M. Correlation between glucocorticoid receptor content and the antiproliferative effect of dexamethasone in experimental solid tumors. Cancer Res., 43: 4757-4761, 1983.

Braunschweiger, P. G., Ting, H. L., and Schiffer, L. M. Receptor-mediated antiproliferative effects of corticosteroids in Lewis Lung Tumors. Eur. J. Cancer Clin. Oncol., 20: 427-433, Nishimura, K., Nonomura, N., Satoh, E., Harada, Y., Nakayama, M., Tokizane, T., Fukui, T., Ono, Y., Inoue, H., Shin, M., Tsujimoto, Y., Takayama, H., Aozasa, K., and Okuyama, A. Potential mechanism for the effects of dexamethasone on growth of androgen-independent prostate cancer. J. Natl. Cancer Inst., 93: 1739-1746, 2001.

Broxmeyer, H., Benningre, L., and Patel, S. Kinetic response of human marrow myeloid progenitor cells to in vivo treatment of patients with granulocyte colony-stimulating factor is different from the response to treatment with granulocyte-macrophage colony-stimulating factor. Exp. Hemat., 22: 100-102, 1994.

Bullen, W. W., Andress, L. D., Chang, T., Whitfield, L. R., Welch, M. L., and Newman, R. A. A high-performance liquid chromatographic assay for CI-973, a new anticancer platinum diamine complex, in human plasma and urine ultrafiltrates. Cancer Chemother. Pharmacol., 30:193-198, 1992.

Carmeliet, P. and Jain, R. K. Angiogenesis in cancer and other diseases. Nature, 407: 249-257, 2000.

Cashman, J. D., Eaves, A. C., and Raines, E. W. Mechanisms that regulate the cell cycle status of very primitive hematopoietic cells in long-term human marrow cultures. I. stimulatory role of a variety of mesenchymal cell activators and inhibitory role of TGF-β. Blood, 75: 96-101, 1990.

Chudgar, U. H., Rundus, C. H., and Peterson, V. M. Recombinant human interleukin-1 receptor antagonist protects early myeloid progenitors in a murine model of cyclophosphamide-induced myelotoxicity. Blood, 85: 2393-2401, 1995.

Coussens, L. M., Werb, Z. Inflammation and cancer. Nature, 420: 860-867, 2002.

Demetri, G. D., Anderson, K. C. Bone marrow failure. In: Clinical Oncology, pp. 443-456. New York: Churchill Livingstone, 1995.

Duffull S B, Robinson B A Clinical pharmacokinetics and dose optimisation of carboplatin. Clin Pharmacokinet 33:161, 1997

Dunlop, D., Wright, E., and Lorimore, S. Demonstration of stem cell inhibition and myeloprotective effects of SCI-rhMIP-Iα in vivo. Blood, 79: 2221-2225, 1992.

Futami, H., Jansen, R., and MacPhee, M. Chemoprotective effects of recombinant human IL-1α in cyclophosphamide-treated normal and tumor-bearing mice. Protection from acute toxicity, hematologic effects, development of late mortality, and enhanced therapeutic efficacy. J. Immunol., 145: 4121-4130, 1990.

Ganser A, Karthaus M. Clinical use of hematopoietic growth factors. Curr Opin Oncol: 265, 1996.

Gorman, A. M., Hirt, U. A., Orrenius, S., and Ceccatelli, S. Dexamethasone pre-treatment interferes with apoptotic death in glioma cells. Neuroscience, 96: 417-425, 2000.

Griffin, J. Hematopoietic growth factors. In: Cancer Principles and Practice of Oncology, 5^{th} Ed., pp. 2639. Philadelphia: Lippincott-Raven, 1997.

Grzegorzewski, K., Ruscetti, F., and Usui, N. Recombinant transforming growth factor β₁ and β₂ protect mice from acutely lethal doses of 5-fluorouracil and doxorubicin. J. Exp. Med., 180: 1047-1057, 1994.

Hashizume, H., Baluk, P., Morikawa, S., McLean, J. W., Thurston, G., Roberge, S., Jain, R. K. and McDonald, D. M. Openings between defective endothelial cells explain tumor vessel leakiness. Am. J. Pathol., 156: 1363-1380, 2000.

Heinemann V. Gemcitabine in the treatment of advanced pancreatic cancer: a comparative analysis of randomized trials. Semin Oncol 29:9, 2002

Jain, R. K. Normalizing tumor vasculature with anti-angiogenic therapy: a new paradigm for combination therapy. Nat. Med., 7: 987-989, 2001.

Jain, R. K. Transport of molecules in the tumor interstitium: a review. Cancer Res., 47: 3039-3051, 1987.

Janik, J., Miller, L., and Smith II, J. Prechemotherapy granulocyte-macrophage colony stimulating factor (GM-CSF) prevents topotecan-induced neutropenia. ASCO Proceedings, 12: 1507, 1993.

Joyce, R., and Chervenick, P. Corticosteroid effect on granulopoiesis in mice after cyclophosphamide. J. Clin. Invest., 60: 277-283, 1977.

Kaufman D, Chabner B A Clinical strategies for cancer treatment: The role of Drugs. In: Cancer Chemotherapy and Biotherapy, ed. Chabner B A, Longo D L, Lippincott-Raven, Philadelphia, Pa., pp 1-16, 1996.

Kerbel, R. and Folkman, J. Clinical translation of angiogenesis inhibitors. Nat. Rev. Cancer, 2: 727-739, 2002.

Kriegler, A., Bernardo, D., and Verschoor, S. Protection of murine bone marrow by dexamethasone during cytotoxic chemotherapy. Blood, 83: 65-71, 1994.

Kristjansen, P. E., Boucher, Y., and Jain, R. K. Dexamethasone reduces the interstitial fluid pressure in a human colon adenocarcinoma xenograft. Cancer Res., 53: 4764-4766, 1993.

Mackal, C. L. T-Cell immunodeficiency following cytotoxic antineoplastic therapy: a review. Stem Cells, 18: 10-18, 2000.

Matsukado, K., Nakano, S., Bartus, R. T., and Black, K. L. Steroids decrease uptake of carboplatin in rat gliomas—uptake improved by intracarotid infusion of bradykinin analog, RMP-7. J. Neurooncol., 34: 131-138, 1997.

Ono, M., Torisu, H., Fukushi, J., Nishie, A. and Kuwano, M. Biological implications of macrophage infiltration in human tumor angiogenesis. Cancer Chemother. Pharmacol., 43 Suppl: S69-71, 1999.

Peters G, Van der Vijgh W. Protection of normal tissues from the cytotoxic effects of chemotherapy and radiation by amifostine (WR-2721): preclinical aspects. Eur J Cancer 31A(Suppl): S1, 1995

Podda S, Ward M, Himelstein A, Richardson C, Flor-Weiss E D L, Smith L, Gottesman M, Pastan I, Bond A. Transfer and expression of the human multiple drug resistance gene in live mice. Proc Natl Acad Sci 89: 9676, 1992

Prasad, G., Wang, H., Agrawal, S., and Zhang, R. Antisense oligonucleotides targeted to MDM2 oncogene as a novel approach to the treatment of glioblastoma multiforme. Anticancer Res., 22: 107-116, 2002.

Rinehart, J., Delamater, E., and Keville, L. Corticosteroid modulation of interleukin-1 hematopoietic effects and toxicity in a murine system. Blood, 84: 1457-1463, 1994.

Rinehart, J., Keville, L., and Measel, J. Corticosteroid alteration of carboplatin-induced hematopoietic toxicity in a murine model. Blood, 86: 4493-4499, 1995.

Rinehart, J., Keville, L., Neidhart, J., Wong, L., DiNunno, L., Kinney, P., Aberle, M., Tadlock, L., and Cloud, G. Hematopoietic protection by dexamethasone of granulocyte-macrophage colony stimulating factor (GM-CSF) in patients with carboplatin and ifosfamide. Am. J. Clin. Oncol., in press.

Rinehart, J. J., and Keville, L. Corticosteroid alteration of carboplatin induced hematopoietic toxicity: comparison of efficacy in normal and hematopoietically impaired tumor bearing mice. Cancer Radiopharml., 2: 101-109, 1997.

Rofstad, E. K., Tunheim, S. H., Mathiesen, B., Graff, B. A., Halsor, E. F., Nilsen, K., and Galappathi, K. Pulmonary and lymph node metastasis is associated with primary tumor interstitial fluid pressure in human melanoma xenografts. Cancer Res., 62: 661-664, 2002.

Teicher, B. A., Herman, T. S., Holden, S. A., Wang, Y. Y., Pfeffer, M. R., Crawford, J. W., Frei E, III. Tumor resistance to alkylating agents conferred by mechanisms operative only in vivo. Science, 247: 1457-1461, 1990.

Torisu, H., Ono, M., Kiryu, H., Furue, M., Ohmoto, Y., Nakayama, J., Nishioka, Y., Sone, S. and Kuwano, M. Macrophage infiltration correlates with tumor stage and angiogenesis in human malignant melanoma: possible involvement of TNFalpha and IL-1alpha. Int. J. Cancer, 85: 182-188, 2000.

Underwood, J. L., Murphy, C. G., Chen, J., Franse-Carman, L., Wood, I., Epstein, D. L. and Alvarado, J. A. Glucocorticoids regulate transendothelial fluid flow resistance and formation of intercellular junctions. Am. J. Physiol., 277: C330-342, 1999.

Vadhan-Raj, S., Broxmeyer, H., and Hittelman, W. Abrogating chemotherapy-induced myelosuppression by recombinant granulocyte-macrophage colony-stimulating factor in patients with sarcoma: protection at the progenitor cell level. J. Clin. Oncol., 10:1266-1277, 1992.

Wang H, Li M, Dubuisson J, Scachinger V, Haslinger M, Rinehart J J, Zhang R. Dexamethasone as a chemoprotectant in cancer chemotherapy: Hematoprotective effects and altered pharmacokinetics and tissue distribution of carboplatin and gemcitabine. Proc. 94^{th} Annual Meeting AACR 2003; Vol. 44, pp. 392 (abstract # 1729).

Wang, C.Y., Mayo, M. W., Korneluk, R. G., Goeddel, D. V. and Baldwin, A. S. Jr. NF-kappaB antiapoptosis: induction of TRAF1 and TRAF2 and c-IAP1 and c-IAP2 to suppress caspase-8 activation. Science. 281: 1680-1683, 1998.

Wang, H., Cai, Q., Zeng, X., Yu, D., Agrawal, S., and Zhang, R. Antitumor activity and pharmacokinetics of a mixed-backbone antisense oligonucleotide targeted to RIα subunit of protein Iinase A after oral administration. Proc. Natl. Acad. Sci. USA, 96: 13989-13994, 1999.

Wang, H., Nan, L., Yu, D., Agrawal, S., and Zhang, R. Antisense anti-MDM2 oligonucleotides as a novel therapeutic approach to human breast cancer: In vitro and in vivo activities and mechanisms. Clin Cancer Res. 7: 3613-3624, 2001.

Wang, H., Nan, L., Yu, D., Lindsey, J. R., Agrawal, S., and Zhang, R. Antitumor efficacy of a novel antisense anti-mdm² mixed-backbone oligonucleotide in human colon cancer models: p53-dependent and p53-independent mechanisms. Mol. Med., 8: 184-198, 2002.

Yamamoto, Y. and Gaynor, R. B. Therapeutic potential of inhibition of the NF-kappaB pathway in the treatment of inflammation and cancer. J. Clin. Invest., 107: 135-142, 2001.

Yu, W. D., McElwain, M. C., Modzelewski, R. A., Russell, D. M., Smith, D. C., Trump, D. L., and Johnson, C. S. Enhancement of 1,25-dihydroxyvitamin D3-mediated antitumor activity with dexamethasone. J. Natl. Cancer Inst., 90: 134-141, 1998.

Zhang, R., Diasio, R. B., Lu, Z., Liu, T., Jiang, Z., Galbraith, W. M. and Agrawal, S., Pharmacokinetics and tissue distribution in rats of an oligodeoxynucleotide phosphorothioate (GEM91) developed as a therapeutic agent for human immunodeficiency virus type-1. Biochem Pharmacol 49: 929-939, 1995.

Zhang, R., Lu, Z., Liu, T., Soong, S.-j., Diasio, R. B. Relationship between circadian-dependent toxicity of 5-fluorodeoxyuridine and circadian rhythms of pyrimidine enzymes: possible relevance to fluoropyrimidine chemotherapy. Cancer Res., 53: 2816-2822, 1993.

1. A method of screening for an agent that increases the efficacy of an antineoplastic chemotherapeutic agent and reduces the hematotoxicity of the chemotherapeutic agent, comprising
a. contacting a test tumor cell with the chemotherapeutic agent and the agent to be screened and detecting a reduction in cell division or an increase in cell death as compared to a control tumor cell contacted with the chemotherapeutic agent in the absence of the agent to be screened, a reduction in cell division or an increase in cell death indicating an agent that increases in the efficacy of the chemotherapeutic agent; and
b. contacting a test hematopoietic cell with the chemotherapeutic agent and the agent to be screened and detecting an increase in cell division or a reduction in cell death as compared to a control hematopoietic cell contacted with the chemotherapeutic agent in the absence of the agent to be screened, an increase in cell division or a decrease in cell death indicating an agent that reduces the hematotoxicity of the chemotherapeutic agent.

2. The method of claim 1, wherein the increase in efficacy of the chemotherapeutic agent comprises an increase in uptake of the chemotherapeutic agent by the test tumor cell as compared to the control tumor cells.

3. The method of claim 1, wherein the reduction in hematotoxicity comprises a decrease in uptake of the chemotherapeutic agent by the test hematopoietic cell as compared to the control hematopoietic cell.

4. The method of claim 1, wherein the tumor cell is a solid tumor cell.

5. The method of claim 1, wherein the tumor cell is a cell from a tumor cell line.

6. The method of claim 1, wherein the hematopoietic cell is a splenic cell.

7. The method of claim 1, wherein the hematopoietic cell is a bone marrow cell.

8. The method of claim 1, wherein the test tumor cell or the test hematopoietic cell is contacted with the agent to be screened prior to contact with the chemotherapeutic agent.

9. The method of claim 1, wherein the test tumor cell or the test hematopoietic cell is contacted wit the agent to be screened daily for four days prior to contact with the chemotherapeutic agent.

10. The method of claim 1, wherein the test tumor cell and the test hematopoietic cell are contacted with the agent to be screened prior to contact with the chemotherapeutic agent.

11. The method of claim 1, wherein the test tumor cell and the test hematopoietic cell are contacted with the agent to be screened daily for three to seven days prior to contact with the chemotherapeutic agent.

12. The method of claim 1, wherein the test tumor cell and the test hematopoietic cell are contacted with the agent to be screened daily for four days prior to contact with the chemotherapeutic agent.

13. The method of claim 1, wherein the contacting steps are in vivo.

14. An agent identified by the method of claim 1.

15. The agent of claim 1, wherein the agent is not dexamethasone.

16. The agent of claim 1, wherein the agent is not cortisone acetate.

17. A method of treating a subject with cancer, comprising the steps of
a. administering daily to the subject the agent of claim 14 for three to seven days prior to administering to the subject an antineoplastic chemotherapeutic agent; and
b. administering to the subject the antineoplastic chemotherapeutic agent.

18. The method of claim 17, wherein the administration step of (a) for four days.

19. A method of screening for an agent that increases the efficacy of an antineoplastic chemotherapeutic agent and reduces the hematotoxicity of the chemotherapeutic agent, comprising
a. administering to a test animal with cancer the chemotherapeutic agent and the agent to be screened;
b. detecting a reduction in cancer cell division or an increase in cancer cell death in the test animal as compared to a control animal with cancer, wherein the control animal is administered the chemotherapeutic agent in the absence of the agent to be screened, a reduction in tumor cell division or an increase in tumor cell death indicating an agent that increases in the efficacy of the chemotherapeutic agent; and
c. detecting an increase in hematopoietic cell division or a reduction in hematopoietic cell death in the test animal as compared to the control animal, an increase in hematopoietic cell division or a decrease in hematopoietic cell death indicating an agent that reduces the hematotoxicity of the chemotherapeutic agent

20. The method of claim 19, wherein the increase in efficacy of the chemotherapeutic agent comprises an increase in uptake of the chemotherapeutic agent by the cancer cells in the test animal as compared to the cancer cells in the control animal.

21. The method of claim 19, wherein the reduction in hematotoxicity comprises a decrease in uptake of the chemotherapeutic agent by the hematopoietic cells in the test animal as compared to the hematopoietic cells of the control animal.

22. The method of claim 19, wherein the test animal and control animal comprise tumor xenografts.

23. The method of claim 19, wherein the cancer cells are solid tumor cells.

24. The method of claim 19, wherein the increase in efficacy of the chemotherapeutic agent, comprises an increase in uptake of the chemotherapeutic agent by the tumor in the test animal as compared to the tumor in the control animal.

25. The method of claim 19, wherein the reduction in hematotoxicity comprises a decrease in uptake of the chemotherapeutic agent by the hematopoietic tissues of the test animal as compared to the control animal.

26. The method of claim 19, wherein the hematopoietic tissue is selected from either bone marrow or splenic tissue.

27. The method of claim 19, wherein the test animal is administered the agent to be screened prior to administration of the chemotherapeutic agent.

28. The method of claim 19, wherein the test animal is administered the agent to be screened daily for three to seven days prior to administration of the chemotherapeutic agent.

29. The method of claim 19, wherein the test animal is administered the agent to be screened daily for four days prior to administration of the chemotherapeutic agent.

30. An agent identified by the method of claim 19.

31. A method of treating a subject with cancer, comprising the steps of
a. administering daily to the subject the agent of claim 30 for three to seven days prior to administering to the subject an antineoplastic chemotherapeutic agent; and
b. administering to the subject the antineoplastic chemotherapeutic agent.

32. The method of claim 31, wherein the administration step of (a) for four days.
